(19) United States
(12) Patent Application Publication
(10) Pub. No.: US 2022/0396837 A1
(43) Pub. Date: Dec. 15, 2022

(54) METHODS AND PRODUCTS FOR MINIMAL RESIDUAL DISEASE DETECTION

(71) Applicants: Genecast (Beijing) Biotechnology Co., Ltd., Beijing (CN); Zhenyue Biotechnology Jiangsu Co., Ltd., Taizhou City (CN); Genecast Biotechnology Co., Ltd., Jiangsu (CN)

(72) Inventors: Yaxi ZHANG, Beijing (CN); Hongyu XIE, Beijing (CN); Weizhi CHEN, Beijing (CN); Ying YANG, Beijing (CN); Rui FAN, Beijing (CN); Xiuyu ZHAO, Beijing (CN); Piao YANG, Beijing (CN); Jianing YU, Beijing (CN); Bo DU, Beijing (CN)

(73) Assignees: Genecast (Beijing) Biotechnology Co., Ltd., Beijing (CN); Zhenyue Biotechnology Jiangsu Co., Ltd., Taizhou City (CN); Genecast Biotechnology Co., Ltd., Jiangsu (CN)

(21) Appl. No.: 17/475,072

(22) Filed: Sep. 14, 2021

(30) Foreign Application Priority Data
Jun. 10, 2021 (CN) 2021106458579

(51) Int. Cl. C12Q 1/6886; C12Q 1/6837; C12Q 1/6806

(52) U.S. Cl. CPC C12Q 1/6886; C12Q 1/6837; C12Q 1/6806; C12Q 2600/158; C12Q 2600/156

(57) ABSTRACT

Methods are disclosed for determining the minimal residual cancer status of an individual utilizing assays that detect cancer associated genetic variation in extracellular DNA. The disclosed methods provide for personalized cancer detection based on the genetic profile of solid cancer tissue of an individual under study. The disclosed methods further provide for noise reduction in the sequencing of extracellular DNA and reduced false positive rates in minimal residual cancer status determination.

### CLAIM OF PRIORITY

This application claims the benefit of priority to Chinese Patent Application No. 2021106458579, filed Jun. 10, 2021, the entire content of which is incorporated herein by reference.

### BACKGROUND OF INVENTION

Circulating tumor DNA (ctDNA) refers to DNA originating from a tumor which may be detected in the circulatory system of the body. In view of its tumor origin, ctDNA exhibits similar genetic variation as the source tumor DNA, in contrast to corresponding non-cancerous genomic sequences. Although ctDNA has a short half-life, it offers benefits for study as it can be easily sampled, in comparison to sampling a solid tumor which commonly requires a biopsy. Therefore, ctDNA can provide an accurate and convenient source of information for medication guidance, drug resistance tracking, and other forms of medical intervention and/or monitoring.

Recently, studies have shown that the prognosis of a patient is related to the clearance of ctDNA from the blood after a cancer treatment protocol, such as drug treatment or surgery. If the ctDNA of a treated patient has cleared, the prognosis of the patient tends to be good. In contrast, if a patient tests positive for residual ctDNA after treatment, even a patient with early-stage cancer tends to have a relatively high recurrence rate and correspondingly poorer prognosis. Thus, the presence of ctDNA may be indicative of the metastasis of micro-tumors in a patient. Studies have shown that the ctDNA of patients signals a recurrent cancer condition much earlier than can be detected by radiology alone. Therefore, ctDNA provides a molecular marker of minimal residual disease (MRD) in a patient. Detection of ctDNA can be used not only to evaluate the effectiveness of treatment and classify recurrence risk, but it can also be used to timely design a personalized follow-up treatment plan, and dynamically monitor cancer recurrence.

Challenges are presented by the need for MRD technology to identify extremely trace amounts of ctDNA signals in the blood. The difficulty lies in how to obtain ctDNA signals more sensitively and determine the authenticity of low-frequency ctDNA signals. In order to obtain ctDNA signals more sensitively, MRD assays are often designed to track numerous genomic sites. Yet, the multi-site assays present challenges of information processing and determination of MRD disease state.

### SUMMARY OF THE INVENTION

The present disclosure provides a set of novel MRD detection and evaluation methods to address the challenges of MRD testing. In certain aspects, the disclosed methods include detection methods based on genetic variation in tumor tissue obtained by the DNA sequencing of a patient's tumor tissue to establish the patient's tumor-specific variation pattern. In certain aspects, only the patient's specific variation pattern is tracked. The disclosed methods substantially eliminate the noise signal in plasma samples caused by clonal hematopoiesis and significantly improves the reliability of subsequent plasma mutation signals.

Additional objects, advantages and novel features of the present disclosure will be set forth in part in the description which follows, and in part will become apparent to those skilled in the art upon examination of the following or may be learned by practice of the disclosed methods. The objects and advantages of the disclosed methods may be realized and attained by means of the instrumentalities and combinations particularly pointed out in the appended claims.

The following numbered paragraphs [0007]-[0039] contain statements of broad combinations of the inventive technical features herein disclosed:

1. A method for determining the minimal residual cancer status of an individual comprising:

a) selecting a panel of loci comprising human genomic regions that may host mutated genes in a particular type of solid tumor;
b) referencing a database of baseline measures of sequence information for the panel of loci;
c) preparing at least one mathematical distribution of sequence information at one or more locus based on the database of step (b), wherein a first portion of the baseline measures at a locus is classified as not exhibiting variation and a second portion of the baseline measures at the locus is classified as exhibiting variation, wherein the second portion of the baseline measures is statistically fitted and combined with the first portion of baseline measures;
d) obtaining tumor sample DNA sequence information collected from a tumor sample from the individual and identifying one or more genomic variants within the selected panel of loci;
e) obtaining extracellular DNA sequence information for the panel of loci from the individual, wherein the sequence information is collected from a plasma sample from the individual, wherein the plasma sample comprises extracellular DNA;
f) comparing the sequence information of step (e) to at least one corresponding distribution of step (c) for one or more genomic variants of step (d), wherein the comparison determines probabilities that differences exist at the one or more genomic variants between the extracellular DNA sequence information of the individual and the corresponding baseline measures of step (b), thereby providing at least one probability of genomic variant level significance;
g) combining the genomic variant level significance probabilities into a combined sample level probability score and
h) determining that the individual has a positive status for minimal residual cancer if the p-value of the combined sample level probability score of step (g) is equal to or less than a threshold value.

2. A method for determining the minimal residual cancer status of an individual comprising:

a) selecting a panel of loci comprising human genomic regions that may host mutated genes in a particular type of solid tumor;
b) referencing a database of baseline measures of sequence information for the panel of loci;
c) preparing at least one mathematical distribution of sequence information at one or more locus based on the database of step (b), wherein a first portion of the baseline measures at a locus is classified as not exhibiting variation and a second portion of the baseline measures at the locus is classified as exhibiting variation, wherein the second portion of the baseline measures is statistically fitted and combined with the first portion of baseline measures;
d) obtaining tumor sample DNA sequence information collected from a tumor sample from the individual and identifying one or more genomic variants within the selected panel of loci;
e) obtaining extracellular DNA sequence information for the panel of loci from the individual, wherein the sequence information is collected from a plasma sample from the individual, wherein the plasma sample comprises extracellular DNA;
f) comparing the sequence information of step (e) to at least one corresponding distribution of step (c) for one or more genomic variants of step (d), wherein the comparison determines probabilities that differences exist at the one or more genomic variants between the extracellular DNA sequence information of the individual and the corresponding baseline measures of step (b), thereby providing at least one probability of genomic variant level significance;
g) combining the genomic variant level significance probabilities into a combined sample level probability score and
h) determining that the individual has a negative status for minimal residual cancer if the p-value of the combined sample level probability score of step (g) is greater than a threshold value.

3. A method for determining the minimal residual cancer status of an individual comprising:

a) selecting a panel of loci comprising human genomic regions that may host mutated genes in a particular type of solid tumor;
b) referencing a database of baseline measures of sequence information for the panel of loci;
c) preparing at least one mathematical distribution of sequence information at one or more locus based on the database of step (b), wherein a first portion of the baseline measures at a locus is classified as not exhibiting variation and a second portion of the baseline measures at the locus is classified as exhibiting variation, wherein the second portion of the baseline measures is statistically fitted and combined with the first portion of baseline measures;
d) obtaining tumor sample DNA sequence information collected from a tumor sample from the individual and identifying one or more genomic variants within the selected panel of loci;
e) obtaining extracellular DNA sequence information for the panel of loci from the individual, wherein the sequence information is collected from a plasma sample from the individual, wherein the plasma sample comprises extracellular DNA;
f) comparing the sequence information of step (e) to at least one corresponding distribution of step (c) for at least one genomic variants of step (d), wherein the comparison determines a probability that a difference exists at the one or more genomic variants between the extracellular DNA sequence information of the individual and the corresponding baseline measures of step (b), thereby providing at least one probability of genomic variant level significance; and
g) determining that the individual has a positive status for minimal residual cancer if the p-value of at least one genomic variant of step (f) is equal to or less than a threshold value.

4. A method for determining the minimal residual cancer status of an individual comprising:

a) selecting a panel of loci comprising human genomic regions that may host mutated genes in a particular type of solid tumor;
b) referencing a database of baseline measures of sequence information for the panel of loci;
c) preparing at least one mathematical distribution of sequence information at one or more locus based on the database of step (b), wherein a first portion of the baseline measures at a locus is classified as not exhibiting variation and a second portion of the baseline measures at the locus is classified as exhibiting variation, wherein the second portion of the baseline measures is statistically fitted and combined with the first portion of baseline measures;
d) obtaining tumor sample DNA sequence information collected from a tumor sample from the individual and identifying one or more genomic variants within the selected panel of loci;
e) obtaining extracellular DNA sequence information for the panel of loci from the individual, wherein the sequence information is collected from a plasma sample from the individual, wherein the plasma sample comprises extracellular DNA;
f) comparing the sequence information of step (e) to at least one corresponding distribution of step (c) for at least one genomic variants of step (d), wherein the comparison determines a probability that a difference exists at the one or more genomic variants between the extracellular DNA sequence information of the individual and the corresponding baseline measures of step (b), thereby providing at least one probability of genomic variant level significance; and
g) determining that the individual has a negative status for minimal residual cancer if the p-value of none of the at least one genomic variant of step (f) is equal to or less than a threshold value.

5. A method for determining the minimal residual cancer status of an individual comprising:

a) selecting a panel of loci comprising human genomic regions that may host mutated genes in a particular type of solid tumor;
b) referencing a database of baseline measures of sequence information for the panel of loci;
c) preparing at least one mathematical distribution of sequence information at one or more locus based on the database of step (b), wherein any variation exhibited by the baseline measures is conformed to a binomial distribution;
d) obtaining tumor sample DNA sequence information collected from a tumor sample from the individual and identifying one or more genomic variants within the selected panel of loci;
e) obtaining extracellular DNA sequence information for the panel of loci from the individual, wherein the sequence information is collected from a plasma sample from the individual, wherein the plasma sample comprises extracellular DNA;
f) comparing the sequence information of step (e) to at least one corresponding distribution of step (c) for one or more genomic variants of step (d), wherein the comparison determines probabilities that differences exist at the one or more genomic variants between the extracellular DNA sequence information of the individual and the corresponding baseline measures of step (b), thereby providing at least one probability of genomic variant level significance;
g) combining the genomic variant level significance probabilities into a combined sample level probability score; and
h) determining that the individual has a positive status for minimal residual cancer if the p-value of the combined sample level probability score of step (g) is equal to or less than a threshold value.

6. A method for determining the minimal residual cancer status of an individual comprising:

a) selecting a panel of loci comprising human genomic regions that may host mutated genes in a particular type of solid tumor;
b) referencing a database of baseline measures of sequence information for the panel of loci;
c) preparing at least one mathematical distribution of sequence information at one or more locus based on the database of step (b), wherein any variation exhibited by the baseline measures is conformed to a binomial distribution;
d) obtaining tumor sample DNA sequence information collected from a tumor sample from the individual and identifying one or more genomic variants within the selected panel of loci;
e) obtaining extracellular DNA sequence information for the panel of loci from the individual, wherein the sequence information is collected from a plasma sample from the individual, wherein the plasma sample comprises extracellular DNA;
f) comparing the sequence information of step (e) to at least one corresponding distribution of step (c) for one or more genomic variants of step (d), wherein the comparison determines probabilities that differences exist at the one or more genomic variants between the extracellular DNA sequence information of the individual and the corresponding baseline measures of step (b), thereby providing at least one probability of genomic variant level significance;
g) combining the genomic variant level significance probabilities into a combined sample level probability score; and
h) determining that the individual has a negative status for minimal residual cancer if the p-value of the combined sample level probability score of step (g) is greater than a threshold value.

7. A method for determining the minimal residual cancer status of an individual comprising:

a) selecting a panel of loci comprising human genomic regions that may host mutated genes in a particular type of solid tumor;
b) referencing a database of baseline measures of sequence information for the panel of loci;
c) preparing at least one mathematical distribution of sequence information at one or more locus based on the database of step (b), wherein any variation exhibited by the baseline measures is conformed to a binomial distribution;
d) obtaining tumor sample DNA sequence information collected from a tumor sample from the individual and identifying one or more genomic variants within the selected panel of loci;
e) obtaining extracellular DNA sequence information for the panel of loci from the individual, wherein the sequence information is collected from a plasma sample from the individual, wherein the plasma sample comprises extracellular DNA;
f) comparing the sequence information of step (e) to at least one corresponding distribution of step (c) for at least one genomic variants of step (d), wherein the comparison determines a probability that a difference exists at the one or more genomic variants between the extracellular DNA sequence information of the individual and the corresponding baseline measures of step (b), thereby providing at least one probability of genomic variant level significance; and
g) determining that the individual has a positive status for minimal residual cancer if the p-value of at least one genomic variant of step (f) is equal to or less than a threshold value.

8. A method for determining the minimal residual cancer status of an individual comprising:

a) selecting a panel of loci comprising human genomic regions that may host mutated genes in a particular type of solid tumor;
b) referencing a database of baseline measures of sequence information for the panel of loci;
c) preparing at least one mathematical distribution of sequence information at one or more locus based on the database of step (b), wherein any variation exhibited by the baseline measures is conformed to a binomial distribution;
d) obtaining tumor sample DNA sequence information collected from a tumor sample from the individual and identifying one or more genomic variants within the selected panel of loci;
e) obtaining extracellular DNA sequence information for the panel of loci from the individual, wherein the sequence information is collected from a plasma sample from the individual, wherein the plasma sample comprises extracellular DNA;
f) comparing the sequence information of step (e) to at least one corresponding distribution of step (c) for at least one genomic variants of step (d), wherein the comparison determines a probability that a difference exists at the one or more genomic variants between the extracellular DNA sequence information of the individual and the corresponding baseline measures of step (b), thereby providing at least one probability of genomic variant level significance; and
g) determining that the individual has a negative status for minimal residual cancer if the p-value of none of the at least one genomic variant of step (f) is equal to or less than a threshold value.

9. The method of any one of aspects 1-4, wherein the fitting is performed by application of a statistical model selected from the group consisting of a beta-distribution, a gamma-distribution, a Weibull-distribution and any combination thereof.

10. The method of any one of aspects 1, 2, 5 or 6, wherein combining the genomic variant level significance probabilities into a combined sample level probability score comprising application of the formula Pₛₐₘₚₗₑ=Cₘ^{k}ΠPᵢ, wherein m of the combination coefficient (C) represents the number of variants tracked and k represents the number of variants that have passed a variant level threshold, wherein only the variant level significance probabilities that have passed the variant level threshold are included in the Pi multiplication.

11. The method of any one of aspects 1 to 10, wherein sequence information for the individual and sequence information comprised by the baseline measures was collected by PCR or hybridization.

12. The method of aspect 11, wherein the sequence information was collected by PCR.

13. The method of aspect 11, wherein the sequence information was collected by hybridization.

14. The method of any one of aspects 1 to 13, wherein the extracellular DNA sequence information for the panel comprises features selected the group consisting of mapping quality, base quality, position depth, variant supported molecules, fragment size, reads pair concordance, distance from the fragment end, and single/duplex consensus.

15. The method of any one of aspects 1 to 13, wherein the sequence information collected from the plasma sample comprises features selected the group consisting of mapping quality, base quality, position depth, variant supported molecules, fragment size, reads pair concordance, distance from the fragment end, and single/duplex consensus.

16. The method of aspect 14, wherein the comparison of step (f) comprises authentication of at least one feature.

17. The method of any one of aspects 1 to 16, wherein step (b) comprises sequence information obtained for a corresponding panel of loci for extracellular DNA from plasma samples from individuals classified as negative for the cancer.

18. The method of any one of aspects 1 to 17, wherein step (b) comprises sequence information obtained by sequencing tumor and plasma samples from individuals having cancer with the same type of solid tumor, wherein mathematical information for genomic variants within the selected panel of loci identified in the tumor is subtracted from mathematical information for genomic variants within the selected panel of loci in corresponding plasma sample to simulate individuals negative for the cancer.

19. The method of any one of aspects 1 to 18, wherein the comparison of step (f) comprises application of a Monte Carlo simulation.

20. The method of any one of aspects 1 to 19, wherein the comparison of step (f) comprises application of a statistical test based on an expectation set by a mathematical distribution in step (c).

21. The method of any of aspects 1 to 20, wherein in step (c), three mathematical distributions of sequence information are prepared, one for each substitution at each base position of the locus.

22. The method of any one of aspects 1 to 21, wherein in step (c) at least one locus exhibits an insertion or deletion and further wherein, one mathematical distribution of sequence information is prepared, one for each insertion or deletion at the locus.

23. The method of any one of aspects 1 to 22, wherein noise is reduced by limiting tracking to tracking of tumor tissue-specific mutations only in plasma.

24. The method of aspect 10, wherein m≥1.

25. The method of any one of aspects 1 to 24, wherein the panel of loci comprises at least one mutation known to be associated with the type of cancer for which minimal residual cancer status is determined.

26. The method of any one of aspects 1 to 25, wherein the cancer is selected from the group consisting of lung cancer, breast cancer, prostate cancer, colon cancer, melanoma, bladder cancer, non-Hodgkin's lymphoma, renal cancer, endometrial cancer, leukemia, pancreatic cancer, thyroid cancer, and liver cancer.

27. The method of any one of aspects 1 to 26, wherein the individual has previously received treatment for cancer.

28. The method of aspect 27, wherein the treatment for cancer was selected from the group consisting of a drug, a radiation treatment, a surgery and any combination thereof.

29. A computer-implemented method for determining the minimal residual cancer status of an individual according to the method of any one of aspects 1, 2, 5 or 6, wherein one or more of steps (b), (c), (f), (g) and (h) are computed with a computer system.

30. A computer-implemented method for determining the minimal residual cancer status of an individual according to the method of any one of aspects 3, 4, 7 or 8, wherein one or more of steps (b), (c), (f), and (g) are computed with a computer system.

31. A program storage device readable by a computer, tangibly embodying a program of instructions executable by the computer to perform the method steps of any one of aspects 1-28.

32. A computing system for determining the minimal residual cancer status of an individual comprising: a memory for storing programmed instructions; a processor configured to execute the programmed instructions to perform the methods steps of any one of aspects 1-28.

33. A non-transitory, computer readable media with instructions stored thereon that are executable by a processor to perform the methods steps of any one of aspects 1-28.

### BRIEF DESCRIPTION OF THE DRAWINGS

FIG. 1 illustrates a work-flow diagram of one aspect of a method for determining the minimal residual cancer status of an individual

FIG. 2 illustrates the minimum detection limit for hotspot variation in PSC1805 (Probit regression).

FIG. 3 illustrates MRD and recurrence status of 27 patients.

### DETAILED DESCRIPTION OF THE INVENTION

While the present disclosure may be applied in many different forms, for the purpose of promoting an understanding of the principles of the disclosure, reference will now be made to aspects illustrated in the drawings and specific language will be used to describe the same. It will nevertheless be understood that no limitation of the scope of the disclosure is thereby intended. Any alterations and further modifications of the described aspects, and any further applications of the principles of the disclosure as described herein are contemplated as would normally occur to one skilled in the art to which the disclosure relates.

As used herein, the term “authentication” refers to variant confirmation by error-suppression filters or/and signal enhancers. In certain aspects, methods for filtering noise and methods for signal enrichment distinguish between real mutations and false positive noise. In certain aspects, selected features are utilized for authentication which features include one or more of mapping quality, base quality, position depth, variant supported molecules, fragment size, reads pair concordance, distance from the fragment end, and single/duplex consensus.

As used herein, the term “baseline” is used to refer to sequence information indicative of the absence of cancer in an individual. In certain aspects, baseline refers to DNA sequence information collected from individuals classified as negative for cancer. In certain other aspects, baseline refers to DNA sequence information representing the absence of cancer in one or more individual by mathematical processing of DNA sequence information from individuals who are classified as positive for cancer.

As used herein, the term “cancer” refers to a disease in which abnormal cells divide without control. In certain aspects, cancer cells can spread from the location in which the cancer develops to other part of the body.

As used herein, the terms “classified”, “classify” and “classification” refer to one or more assignment to a particular class or category based on aspects of the subject matter classified. In certain embodiments, the aspects of data classified relate to the level of variation found in data and classification of the data based on the level of variation.

As used herein, the term “ctDNA” or “circulating tumor DNA” refers to DNA originating from a tumor which is present in the circulatory system of an individual.

As used herein, “distance from fragment end” refers, for any particular nucleic acid fragment of a given length, to the position of a feature (e.g., a mutation) on the fragment as defined by the distance from the 5′ and 3′ ends of the fragment.

As used herein, the term “distribution” or “mathematical distribution” refers to conversion of nucleic acid sequence information into a numerical format. In certain aspects, nucleic acid sequence information is converted to one or more than one mathematical distribution, which may be in the form of one or more graphs.

As used herein, “extracellular DNA” or “ecDNA” or “cfDNA” refers to any DNA present in an individual which is located outside the cells of the individual. In certain aspects, extracellular DNA is found in the plasma of an individual. In certain further aspects, extracellular DNA derives from the nuclear DNA of an individual. In certain further aspects, extracellular DNA derives from the mitochondrial DNA of an individual.

As used herein, the term “feature” refers to a characteristic which is descriptive of sequence information obtained from one or more individuals. In certain aspects, a features can include one or more of mapping quality, base quality, position depth, variant supported molecules, fragment size, reads pair concordance, distance from the fragment end, and single/duplex consensus.

As used herein, the term “fragment size” refers to the number of nucleic acid bases comprising a sequence of bases.

As used herein, “genomic region” refers to a region of the human genome which is considered of interest. In certain aspects, a genomic region may encompass a single gene of interest, optionally including regulatory regions and regions of unknown function. In certain aspects, a genomic region may encompass multiple known genes as well as regulatory regions and regions of unknown function.

As used herein, “genomic variant” or “variant” refers to any nucleic acid sequence variation observable in a comparison between at least one set of sequence information. In certain aspects, a genomic variant is a variation between the sequence of a gene in a cancer negative baseline and a corresponding gene in an individual for which a cancer diagnosis is performed. In certain aspects, a genomic variant is indicative of a positive cancer status.

As used herein, the term “locus” or “loci” refers to one or more physical locations within the genome of an individual or corresponding locations among individuals. In certain aspects, a locus encompasses a genomic region which is associated with known cancer-causing mutations. In certain aspects, a locus may encompass a genomic region which is not known to be associated with cancer causing mutations.

As used herein, “mapping quality” refers to a determination regarding the probability that a read is misaligned relative to a sequence under study. A higher mapping quality score corresponds to a lower probability of a sequence read being misaligned. In certain aspects, a determination of mapping quality is based on a Phred score defined by the following equation MAPQ=−10(log₁₀ϵ), wherein the ϵ is the estimated probability of misalignment.

As used herein, “minimal residual cancer status” or “residual cancer status” or “minimal residual disease status” or “MRD” refers to a determination or diagnosis of the status of an individual with respect to the presence or absence of cancer cells in the body of the individual. In certain aspects, the minimal residual cancer status of an individual may be positive, but the individual may have no known tumor tissue. In certain aspects, positive minimal residual cancer status indicates cancer cells present in the body of an individual, after the individual has received one or more cancer treatment or therapy.

As used herein, “mutated gene” or “mutant gene” refers to a gene which has a DNA sequence which is different from the corresponding DNA sequence in a majority of individuals classified as not having cancer. In certain aspects, a mutated gene is indicative of the presence of cancer in an individual. In certain further aspects, a mutated gene is found in at least one tumor cell from an individual. In certain aspects, more than one mutant gene is found in at least one tumor cell from an individual.

As used herein, “panel” refers to a group encompassing as few as one member or a large number of members. In certain aspects, a panel of loci refers to one or more locus. In certain further aspects, a panel of loci refers to multiple genomic regions of interest.

As used herein, “position depth” refers to the number of nucleic acid base positions covering a mutation site. In certain aspects, the number of nucleic acid base positions within a mutation site is identified by sequencing of a test sample.

As used herein, the term “read” refers to collection of sequence information. In one aspect, read refers to collection of sequence information from one genomic region. In another aspect read refers to collection of sequence information at more than one genomic region. In certain aspects, read refers to collection of baseline sequence information. In certain aspects, read refers to collection of sequence information from a test sample.

As used herein, “reads pair concordance” refers to the consistency of variation information in a repeated region measured by a read_pair. In one aspect, pair-end sequencing can be performed providing sequence information for the same polynucleotide fragment from opposite directions, 5′ to 3′ a first read (i.e. Read 1) and 3′ to 5′ a second read (i.e. Read 2). In such aspect, the disagreement of Read1 and Read 2 provides an indicator of sequencing noise.

As used herein, “sample level significance” refers to a mathematically combined probability, based on the presence of more than one genomic variant in a sample from an individual, which combined probability may be indicative of the presence of cancer in the sample from the individual. In certain aspects, sample level significance is assessed by tracking a single variant signal (e.g when the tumor tissue has only one traceable variant). Such that, sample_level_significance can be interpreted as a significance assessment of whether the sample is MRD+ based on the information of all the variations tracked in the sample.

As used herein, “sequence information” refers to any nucleic acid sequence information relating to one or more individual. In certain aspects, sequence information relates to DNA sequence information relating to the genome of an individual. In certain aspects, sequence information relates to DNA sequence information from the genome of more than one individual, optionally representing a control group. In certain aspects, sequence information relates to mRNA information from an individual. In certain aspects, sequence information relates to mRNA information from more than one individual, optionally representing a control group. In certain aspects, sequence information is gathered from DNA obtained from an individual classified as cancer negative. In certain other aspects, sequence information is gathered from tumor tissue of an individual. In certain aspects, sequence information is collected directly from cells of an individual. In certain aspects, sequence information results from mathematical calculations based on sequence information from one or more individuals. For example, sequence information may be derived from mathematical removal of variants found in the tumor DNA of an individual from variants found in the sequence information of ecDNA of the same individual.

As used herein, “sequence quality” refers to a level of confidence regarding whether the correct nucleic acid bases are identified at the correct base positions. Accuracy of identification of an individual nucleic acid base at a particular position is referred to as “base quality”. In certain aspects, the sequence quality score is defined by the following equation: Q=−log₁₀(e), where e is the estimated probability of any individual base identification being incorrect.

As used herein, “single consensus” refers to the sequence concordance among family members grouped by unique molecular identifiers (UMIs), which are PCR replicates from the same strand of the same individual polynucleotide.

As used herein, “duplex consensus” refers to the sequence concordance among family members grouped by unique molecular identifiers (UMIs), between the two single-strand-consensus-sequences (SSCS) derived from the two strands of the same individual double-stranded DNA molecule.

As used herein, the term “threshold” refers to a maximum or minimum level designated as a cut-off upon which a determination is based with respect to the cancer status of an individual.

As used herein, “tumor” refers to an abnormal mass of tissue that forms when cells grow and divide more than they should or do not die when they should.

As used herein, “variant supported molecule” refers to, in the case of a particular variant, nucleic acid bases within a mutation site which are indicative of the variant. In certain aspects, the variant support molecule is determined by sequencing of a test sample. In certain aspects, variant support molecule refers to the number of cfDNA molecules that support a specific mutation. The number of molecules can be obtained by combining sequencing data with a deduplication algorithm.

As used herein, “variant level significance” refers to a probability that the presence of a particular genomic variant is indicative of the presence of cancer in an individual. In certain aspects, variant level significance refers to the probability that the calculated variation comes from a baseline noise. The calculation can be based on the variation signal obtained by cfDNA detection, and a mathematical model of its corresponding baseline signal.

The present disclosure provides a set of novel MRD detection and evaluation methods to address the challenges of MRD testing. In certain aspects, the disclosed methods include detection methods based on genetic variation in tumor tissue obtained by the sequencing of a patient's tumor tissue in order to establish the patient's tumor-specific variation pattern. In certain aspects, only the patient's specific variation pattern is tracked. The disclosed methods substantially eliminate the noise signal in plasma samples caused by clonal hematopoiesis and significantly improves the reliability of subsequent plasma mutation signals.

Further disclosed herein are methods for two-level confidence analysis by applying algorithms on variation signals found in a patient's blood that match the genetic variation mapped from an individual's tumor. In certain aspects, a significance analysis is performed by comparing an individual's sampled genetic variation signal with a baseline signal of a cancer negative population, to obtain site-level confidence Pᵥₐᵣᵢₐₙₜₛ. A smaller Pᵥₐᵣᵢₐₙₜₛ indicates a more significant difference, and a higher possibility of a non-noise basis for the signal. Subsequently, a sample-level analysis can be performed. In certain aspects, the genetic variation pattern of a patient may comprise multiple genetic variants for which is obtained a comprehensive confidence level (Pₛₐₘₚₗₑ) at the sample level through joint probability confidence analysis. A smaller Pₛₐₘₚₗₑ represents a greater difference between the variant signal in the patient's blood sample and a baseline population, and a higher probability of ctDNA. In certain aspects, a determination of MRD status of a patient can be based on the confidence level at the sample level.

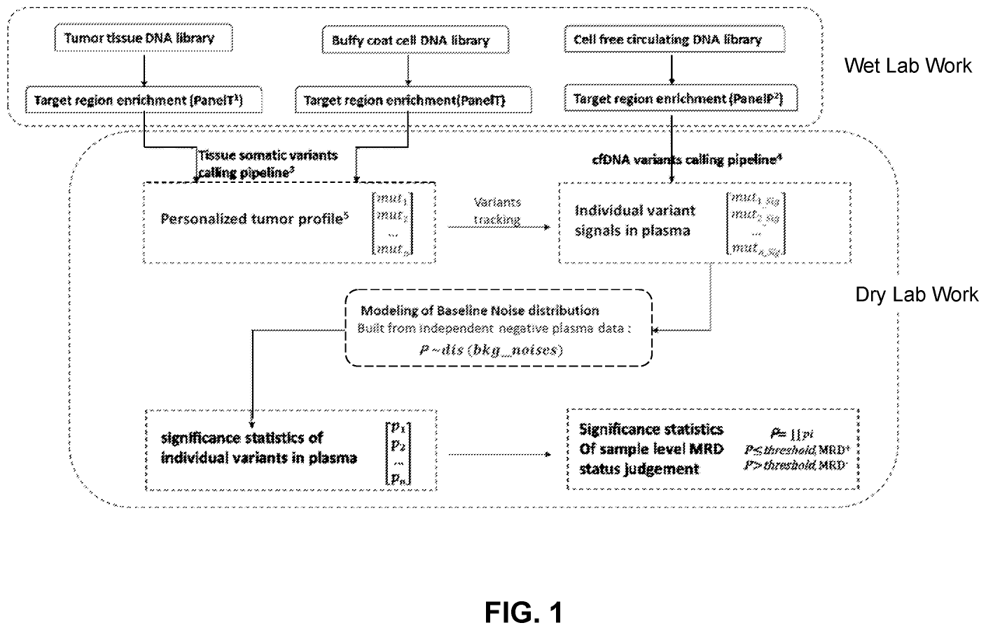

FIG. 1 illustrates one aspect of the presently disclosed method for determining the minimal residual cancer status of an individual. As shown in FIG. 1, PanelT is used to enrich the target region of tumor tissue libraries and matched buffy coat cell DNA libraries and PanelP is used to enrich the target region of plasma DNA libraries. In certain aspects, the enrichment region of PanelP is the same as PanelT. In certain aspects, the enrichment region of PanelP is a subset of PanelT. In certain aspects, PanelP is customized to target only tumor variants as detected in matched tissue. In certain further aspects, negative plasma baseline samples are operated by the same experimental process with the same panelP. Tissue somatic variants calling pipeline: refers to bioinformatic mutation identification based on the sequencing data of tumor tissue and paired buffy coat cell. There are no restrictions on the algorithms or software that may be used with the presently disclosed methods. Paired-calling mode can be applied by matching tumor tissue data and matched blood cell data, or variants can be identified separately from tissue and blood and then the results combined. There are also no restrictions on the mutation filtering rules that may be applied to the presently disclosed methods.

As used in FIG. 1, cfDNA somatic variants calling pipeline: refers to bioinformatic mutation identification based on the sequencing data of cell-free-DNA. There are no restrictions on the variant identification algorithm or software used here, and no restriction on the variant correction rules which can be applied. In certain preferred aspects, the same bioinformatic methods and criteria are applied for the baseline data.

As used in FIG. 1, personalized tumor profile: refers to a patient's personalized collection of tumor-specific variations. In certain aspects, only the variants of this collection in plasma are tracked and provide basis for a determination of the MRD status of an individual.

In certain aspects, disclosed herein are methods for determining the genetic variant signature of a tumor of an individual and the application of the signature to track the residual ctDNA signal in the blood of the individual which provides for the reduction of false positive signals from clonal hematopoiesis and other noise sources.

In certain aspects, not only functional hotspot mutations are tracked, but also clonal non-functional mutations (including synonymous mutations) are tracked simultaneously. In certain aspects, the types of mutations include single nucleotide mutations (SNP), insertion deletion mutations (Indel) and structural mutations (SV). In certain aspects, tracking of multiple variant signals and multiple variant types simultaneously provides more sensitive ctDNA detection.

In certain aspects, the genomic variant signal of an individual is compared to a baseline database constructed from the sequence information from a large cancer negative population group to arrive at a variant level probability or a sample level probability. In some aspects, for each possible variant signal at each genomic locus of interest analyzed, the distribution of the cancer negative population is established through model fitting, and the significance of the variant signal intensity of the patient in analyzed in comparison to the cancer negative population.

In certain aspects, multi-site joint confidence probability analysis is applied to accurately determine a patient's MRD status. Such joint use of multiple sites or sample level probability avoids the problem of reduced assay specificity caused by the increased number of variants tracked and can in certain circumstance provide a more accurate determination of MRD status.

Negative population baseline database: In certain aspects, in the analysis of the variation signal from a plasma sample the database of baseline measures can comprise unadjusted original values or, alternatively, can comprise baseline measures which have been adjusted by application of one or more algorithm to the original values.

In certain aspects, the negative population baseline database is utilized to analyze the significance of a patient's plasma variation signal compared with the negative population's baseline variation signal to identify the presence of ctDNA. In certain preferred aspects, the variation signal of the cancer negative population is obtained through the same experimental procedure and analysis process (conventional MRD coincidence detection) as the patient sample. The distribution of the signal variation may, in some circumstance, be considered distribution of noise.

Preparation of the noise baseline of the negative population database: In certain aspects, for each possible variant signal at each site analyzed, the signal intensity is extracted in the negative population, and established as a model to fit the distribution pattern of the negative population. Such modelling can consist of two parts: 1) the frequency of the population with undetected mutations for specific mutations at specific sites; 2) the distribution model fitting of the detected mutation signals (including but not limited to Beta-distribution, Gamma-distribution, Weibull-distribution and other models).

Data source of the negative population baseline database: In certain aspects, to increase the performance of the MRD status evaluation, the negative population baseline database is required to meet certain conditions, wherein the number of individuals in the baseline database population is larger than a minimum size. In certain aspects, the baseline population size is greater than 1000 individuals.

In certain aspects, the baseline database contains sequence information from the extracellular DNA of cancer negative individuals which has been processed for noise reduction through corresponding deep sequencing of paired white blood cells and deduction of the interference of clonal hematopoietic signals.

In certain aspects, a baseline database can be developed and noise reduced by obtaining sequence information from the extracellular DNA of an individual and subtracting sequence information obtained by sequencing a tumor sample from the individual.

In certain aspects, noise in a baseline database can be reduced by elimination of outliers. Outliers can be caused by operating procedures or other reasons (such as incomplete ctDNA subtraction). The methods disclose herein provide for reduction of noise in the baseline database caused by outliers by removal of outliers in the data.

In certain aspects, a baseline database is used to analyze the confidence level of a single variant signal in a plasma sample from an individual. In one aspect, for a single variant signal in plasma, a large sample size (N, N≥1000) sampling simulation can be performed according to the distribution characteristics of the variant in the baseline database. The frequency of the population not detected with the mutated signals can be extracted and a model built for the vaf of the mutated signal. By applying Monte Carlo simulation, N×Percent (vaf=ZERO) number of zero can be generated. From the distribution model of vaf, N×(1−Percent (vaf=ZERO)) times sampling is performed, so that a plurality of vaf with a total number of N is obtained. By using the N number of vaf as priori noise distribution frequencies respectively, the probability of the signals (VSM, TSM) detected in patients' plasma by using binomial model is calculated, the probability Pi=1−binomial(n≤VSMⱼ−1|TSMⱼ,vafᵢ). Subsequently, a value P_average is used, providing an average value of N number of P values, as the confidence level of this signal variant. A lower P_Average indicates that, the signal variant has a larger difference from the noise of negative baseline population, such that the variant signal of the extracellular DNA is more reliable.

Use of joint confidence probability analysis to determine the MRD status of an individual patient sample. Joint confidence probability analysis, as disclosed herein, provides simultaneous tracking of all the mutations of an individual's personalized tumor-specific variation pattern to determine the individual's MRD status. One of the challenges presented by analysis to determine a MRD positive status is the problem of false positive determinations caused when performing multiple comparisons. In certain aspects, no upper limit is set on the number of variants to be tracked to achieve the highest sensitivity ctDNA signal detection within the allowable range.

Application of sample level probability analysis. In the tumor variation pattern of an individual comprising M number of variations, the M number of variations in the blood can be tracked, and the M number of P values can be obtained based on confidence analysis of the M number of variation signals by applying the aforementioned methods. Among the M number of P values, k number of P values satisfy that P≤Psite_cutoff (confidence threshold for a single variation signal). In this way, the joint confidence probability that is detected is Pₛₐₘₚₗₑ=Cₘ^{k}ΠPᵢ (Pi are k number of variation signals that are below the threshold). When Psample≤Psample_cutoff, the sample is determined to be from an MRD positive individual. In certain aspects, the confidence threshold for a variant or a sample can be 0.05, less than 0.05, 0.04, less than 0.04, 0.03, less than 0.03, 0.02, less than 0.02, 0.01, less than 0.01, 0.005, less than 0.005, 0.004, less than 0.004, 0.003, less than 0.003, 0.002, less than 0.002, 0.001, or less than 0.001.

In certain aspects, in the formula, Pₛₐₘₚₗₑ=Cₘ^{k}ΠPᵢ, m is the number of variants that can be tracked by tumor tissue sequencing, k is the number of P values of the variants that meet the variant_level_significance threshold, and K can be 0, 1, 2 . . . . In certain further aspects, when using the aforementioned formula, m only needs to be greater than or equal to 1. In certain aspects, when m=1, it is a single point decision. In some aspects, when k=0, it is equivalent to that all the mutations tracked in the plasma do not give a significant signal, and one can directly determine MRD-; when k≥1, a value of Psample will be obtained, and the Psample value will be compared with the sample_level threshold to determine the MRD status.

Rich tracking variant types: Variation types as analyzed herein include but are not limited to single nucleotide mutations (SNP), insertions or deletions (Indels) and structural variations (SVs). Simultaneous tracking of multiple types of mutations enables more sensitive ctDNA detection.

Tracking not only functional hotspot mutations, but also other clonal free-riding mutations: This kind of free-riding mutation occurs in the early stage of a tumor. Due to the low evolutionary selection pressure it receives, it will stably exist in the later tumor evolution, which is beneficial to MRD signal tracking as disclosed herein.

### EXAMPLES

The following examples are presented in order to more fully illustrate some embodiments of the invention. They should in no way be construed, however, as limiting the broad scope of the invention. Those of ordinary skill in the art can readily adopt the underlying principles of this discovery to design various compounds without departing from the spirit of the current invention.

### Example 1—Technical Process

### Wet Lab Work

1. A patient's tumor tissue and paired germline cells are sequenced for construction of patient specific sequence information, potentially comprising one or more variant. The goal is to obtain the patient's personalized tumor mutation map, wherein the panel used for enrichment in the target area is panelT (panelTissue).

2. The blood cell-free DNA (cfDNA) of the patient's MRD monitoring point is sequenced. Only mutations of tumor tissue are tracked. If there are only 10 mutations in the tumor tissue, then only those 10 mutations are tracked in the blood sample of the patient. The goal is to track existence of ctDNA in the blood that contains the mutation information based on the patient's tumor mutation map (obtained from the tumor tissue sequence in the previous step). If the ctDNA contains tumor mutations, the MRD status is determined as positive. If the ctDNA does not contain tumor mutations, the MRD status is determined as negative. The panel used to enrich in the target area herein is panelP (panelPlasma).

A “panel” is a collection of selected genomic loci used in the wet lab process which is designed to capture specific genomic regions of interest.

### Dry Lab Work

1. A baseline population database is prepared (can include more than 1000 cancer negative plasma samples. Enrichment: if there is a DNA sample, hybridization of panel, selection of the region of interest in the sequence for study, usually region related to the tumor.) cfDNA mutation signal in the negative population is considered from background noise. cfDNA mutation information is detected in the large-base negative population and the specific mutation are targeted at each site within the coverage of panelP to perform model fitting of background noise.

Thus, for each genomic variant, there is provided a background database (baseline). For a particular variant, 1 of N personalized tumor variants is identified. For each of the N variants, the background database is referenced for comparison to the particular variant in the background (in cases where the plasma sequence of the patient stands in the background database, sequence information is reviewed for being above a threshold or below a threshold). Monte Carlo simulation on a binomial distribution is performed, for example 1000 times, and is used to calculate the variant level probability (to determine if the read is a background noise or a true signal). A sample level probability is a combined probability calculation based on the individual variant level probabilities.

2. Establish a patient's personalized tumor mutation map: obtained through somatic variants calling pipeline of bioinformatics, wherein the parallel construction of paired germline cells eliminates the interference of germline mutations. This pipeline can be any somatic mutation calling method, including different software and algorithms, different threshold settings, different filter condition settings, etc. It also includes different methods of deducting germline mutations, such as using paired calling, or separate calling then filter the germline variations.

3. Tracking tumor-specific mutations in the blood: the tumor-informed method is adopted, that is, only specific mutations at specific sites detected in the tissue are tracked in the blood. The pipeline of blood somatic variants can also be any method used for ctDNA somatic variants calling, including different software and algorithms, different threshold settings, different filter condition settings, etc.

4. Perform single site confidence analysis on the variant signal detected in the blood: track each variant in the patient's tumor variant map in the blood. If the variant is not detected, the variant in the map is negative in the blood. If the variant is detected in the blood, a positive determination cannot immediately be made. First, the possibility that it comes from background noise is evaluated. The method is to analyze the significance of the signal intensity of each variant with the back-noise distribution fitted by the model in the baseline database. When the P-value is particularly small, it indicates that the probability of it coming from background noise is low.

5. Multi-site joint confidence analysis of the variant signals detected in the blood: when multiple variants are tracked at the same time to determine existence of blood ctDNA, multiple single-site confidence analyses are performed; in order to control false positives caused by multiple comparisons, joint confidence analysis is used to ensure the specificity of the MRD assay. This procedure solves the problem found in other methods that the more sites tracked, the worse the specificity becomes.

Special emphasis: the baseline population database is based on the plasma data of the negative population, and its experimental procedures (including the wet and dry lab work) need to be consistent with the DNA operating procedures for the individual patent's sample, such that the baseline can represent the background noise of the overall process. Similarly, while various methods and rules for cfDNA variant-calling can be applied, the calling process and discrimination criteria of the plasma variant signal of the negative population for constructing the baseline database need to remain consistent with the calling process and discrimination criteria of the patient's plasma variant signal analysis. To extend, in order to improve the detection accuracy, the existing literature uses various features to correct the detected variant signals, such as filtering through base quality/read quality, filtering using unique molecule identifiers (UMI), and filtering by conditions such as chain preference, blacklist, edge effect, etc. As another example, when the mutation has the characteristics of Double strand consensus, the confidence of the mutation can be improved.

Features and conditions are compatible with the ctDNA determination method based on the baseline population database can be chose for use when detecting negative populations and patient plasma mutations. Different filtering conditions and correction methods can be used, as long as the same rules are applied to the plasma data of the baseline population and the individual to be tested. Follow-up baseline construction and significance analysis can be performed on the variant signals obtained after applying the rules.

### Example 2—Baseline Population Data

Function: obtaining information of variants from plasma of negative population based on the same technology platform; building the noise model; and conducting significance analysis of the variant signal of the patient's plasma with respect to the noise signal of the negative population to assess possibilities of ctDNA existence.

Requirements: In order to ensure the performance of the test, the negative population baseline database must meet certain conditions, that the size of the population is large enough to meet the establishment of the population distribution model of loci-level variation (≥1000). In addition, the processes applied to the negative population baseline database should be consistent with the processes applied to the plasma of the patient to be tested.

Data collection: Contains the cfDNA data of the tumor patient. Similarly, the data subtracts the noise caused by clonal hematopoiesis by sequencing the white blood cell DNA, and also subtracts the ctDNA signal in the blood by sequencing the tissue of the tumor patient.

Elimination of outliers in the baseline database of negative populations. In order to remove the influence of outliers caused by operating procedures or other reasons (such as ctDNA incomplete subtraction) on the model, treatments are performed to outliers in the data.

Filtering of variation signals of somatic cells of negative population may involve multi-layered methods and combinations thereof. In certain aspects, the extracellular DNA sequence information for the panel comprises features selected the group consisting of position depth, variant supported reads, sequence quality, mapping quality and any combination thereof. Variation information (TSM, VSM) is obtained of all reported loci of each baseline individual within the reporting range, and further integrate individual variation signals to establish a baseline data model.

### Example 3—Baseline Data Model Construction

Algorithms 1 and 2 respectively correspond to two sets of model-building methods and calculation methods of single point variation P values:

Algorithm 1:

According to simulated distribution of the noise signal (VAF, Variant Allele Frequency, VAF=TSM/VSM) in the population based on the established combined model, to estimate probability of patent's plasma variation signal being a noise signal based on model sampling (1) or expected value of the model (2).

Detailed Description: The combined model consists of two parts: 1) a proportion of the population without variation (P_{ZERO}); 2) a fitted model of vaf distribution for a population with variation, the fitted model P_{vaf}˜DIS (vaf) (the fitting models used include, but not limited to Beta-distribution, Gamma-distribution, Weibull-distribution and other models);

Based on the established combined model, two methods may be implemented to conduct significance analysis of single loci variants for plasma:

(1) Based on the model sampling: Conducting Monte Carlo samplings based on the combined model; conducting a statistical calculation to each vaf sample, which is used as a frequency parameter for a binomial distribution; and finally integrating all the statistical results. According to position information of plasma variant locus, calling a combined model for the locus; performing N times sampling (N≥5000) by applying Monte Carlo Simulation, to generate N×P_{zero} number of 0s; meanwhile generating N×(1−P_{ZERO}) number of random VAFs by the variant model [of the combined model]; applying each of the N number of VAFs as a priori noise frequency, to calculate based on a binomial distribution the probability of variant signals (VSM, TSM) of patient's plasma being a noise signal Pi=0, if vafᵢ=0; Pi=1−binomial(n≤VSMⱼ−1|TSMⱼ,vafᵢ), if vafᵢ≠0; combining N number of calculation results, and further calculating an average value of Pi P=Σ₁^{N}Pi to measure the significance level of single point variant in patient's plasma. The lower P is, the greater the difference between the single point variant of the patient's plasma and the negative population baseline noise is, that is, the more likely it is the origin of the ctDNA.

(2) Based on the expected value of the model: Substituting the expected value of the combined model as a parameter into the model, and calculating the significance level of variation of the test plasma. According to the position information of the plasma variant locus, calling a combined model for the locus, wherein expected value of vaf for the population without variants is 0, and the weight is the proportion of the population (P_{zero}), and the expected value of vaf for the population with variants is E(P), and the weight is 1−P_{zero}. As such each of the expected values for the two models may be used to calculate probability of variation signals (VSM, TSM) of patient's plasma from a noise signal respectively. Then the significance level of variant signals of patient's plasma may be measured by calculating a weighted average of the above-calculated probabilities, Pⱼ=(1−P_{zero})*(1−binomial(n≤VSMⱼ−1|TSMⱼ,E(P))). The lower P is, that is the greater the difference between the single point variant of the patient's plasma and the negative population baseline noise is, therefore, the more likely it is the origin of the ctDNA.

Algorithm 2

Build a binomial distribution model based on probability of noise occurrence of θₙₒᵢₛₑ which is implemented as a parameter to a binomial model. Estimate the model parameter θₙₒᵢₛₑ for the noise signal by applying a statistical method (e.g., likelihood estimation, etc.). Then estimate the probability of variant signal of patient's plasma being a noise signal through the complete model assessment.

Detailed description: This model is a single model (not a combined model). Plasma noise signal (VSM, TSM) for a specific variation for a particular loci conform to a binomial distribution in which the probability of noise occurrence θₙₒᵢₛₑ is a parameter, P˜binomial (VSM, TSM, θₙₒᵢₛₑ). The probability of noise occurrence θₙₒᵢₛₑ or the distribution of θₙₒᵢₛₑ, that is f(θₙₒᵢₛₑ), may be approximated based on noise data of baseline population through likelihood estimation L(θₙₒᵢₛₑ|VSM, TSM)=Π₁ⁿbinomial (VSMᵢ, TSMᵢ, θₙₒᵢₛₑ).

Based on the estimated parameters, the probability of variant signals of patient's plasma being a noise signal may be calculated based on the binomial distribution model,

P=1−binomial(n≤VSMⱼ−1|TSMⱼ,θₙₒᵢₛₑ), or

P=1−binomial(n≤VSMⱼ−1|TSMⱼ,f(θₙₒᵢₛₑ)),

where P is used to measure the significance level of variant information in patient's plasma. The lower P is, that is the greater the difference between the single point variant of the patient's plasma and the negative population baseline noise is, therefore, the more likely it is the origin of the ctDNA.

### Example 4—Performance Analysis of Hot-Spot-Driven Single Variant Detection by Combined Model Monte Carlo Sampling Algorithm

This embodiment verifies the sensitivity and specificity of the Combined model Monte Carlo sampling algorithm for hot-spot-driven single variant detection, by analyzing the experimental data for performance verification. In the performance verification experiment, UMI molecular tag adapter was used to construct the library, and then PanelP1 was used (Table 5) to enrich the target region. The PanelP1 covers an interval of 108Kb of 29 genes. The enriched library was sequenced at a high depth. In the sensitivity evaluation, positive sensitivity control-PSC1805 (see Table 1.1 for details), a newly disclosed collection containing 12 known hot-spot-driven variants, was used. 149 healthy people's cfDNA were used for specificity evaluation, in which specificity for detecting 19 tumor hotspot-driven variants was evaluated.

TABLE 1.1
hot-spot variants and ddPCR frequencies in the PSC1805
PSC1805 hot-spot-driven variants information

| | | chromo- | | | | Amino acid | ddPCR |
|---|---|---|---|---|---|---|---|
| # | gene | some | Coordinates | Ref | alt | variation | frequency (%) |
| 1 | BRAF | chr7 | 140453136 | A | T | V600E | 0.92 |
| 2 | EGFR | chr7 | 55241707 | G | A | G719S | 0.94 |
| 3 | EGFR | chr7 | 55242464 | AGGAAT | A | E746_A750del | 1.53 |
| | | | | TAAGAG | | | |
| | | | | AAGC | | | |
| 4 | EGFR | chr7 | 55249005 | G | T | S768I | 1.37 |
| 5 | EGFR | chr7 | 55249071 | C | T | T790M | 0.88 |
| 6 | EGFR | chr7 | 55259515 | T | G | L858R | 1.11 |
| 7 | KRAS | chr12 | 25398285 | C | T | G12S | 0.75 |
| 8 | KRAS | chr12 | 25398284 | C | T | G12D | 0.83 |
| 9 | NRAS | chr1 | 115258747 | C | T | G12D | 0.72 |
| 10 | NRAS | chr1 | 115256530 | G | T | Q61K | 0.76 |
| 11 | NRAS | chr1 | 115256529 | T | C | Q61R | 0.8 |
| 12 | PIK3CA | chr3 | 178952085 | A | G | H1047R | 0.89 |

1.1 Sensitivity and Lowest Detection Limit of Combined model Monte Carlo sampling algorithm

1.1.1 Sample information—The genome of the normal diploid cell line GM12878 was serially diluted with PSC1805. The series of samples of PSC1805 includes 5 dilution gradients. According to the theoretical variation frequency of the hotspot variations, the mean values from high to low are 1%, 0.3%, 0.1%, 0.05% and 0.02%. The 5 gradient samples are named PSC1805-1P, PSC1805-03P, PSC1805-01P, PSC1805-005P and PSC1805-002P, respectively.

1.1.2 Experimental procedure—Firstly, Covaris was used to fragment the five diluted DNA samples of PSC1805-1P, PSC1805-03P, PSC1805-01P, PSC1805-005P and PSC1805-002. Secondly, 30 ng of a fragmented DNA sample was taken and a library constructed by using a KAPA Hyper Preparation Kit. UMI adapters were used in the library construction process. Thirdly, the constructed library was captured using PanelP1 for the target area. The process was repeated three times for each gradient sample. Fourthly, sequencing was performed by using a Novaseq machine. The Novaseq was set to a paired-end sequencing (150PE) to the sample, and the data volume was set to be 8G. The average off-machine sequencing depth was about 40,000×.

1.1.3 PanelP1 baseline model construction: The construction of the baseline model was based on the plasma free DNA data of 1,000 negative populations. The experimental procedures such as construction, capture, and computerization of the plasma library and the amount of data on the computer were fully consistent with the aforementioned standards. Before constructing the model, subtraction of germline mutations and clonal hematopoietic mutations was first performed. In particular, when the data came from tumor patients, tumor tissue-specific mutations were also subtracted. Then, outlier processing was performed to reduce noise, and the remaining variation represented the noise signal of each variation direction (Subtype) of each chromosome coordinate (Position). In this example, the combined model was used to fit the baseline noise signal model, record the proportion of non-variant populations corresponding to each variation direction (Subtype) of each chromosome coordinate (Position), and simulate vaf of the variant population by applying Weibull distribution.

1.1.4 Bioinformation analysis: Since, the DNA fragments in the to-be-tested sample carry the molecular tag adapters in advance, the molecular tags were extracted in the paired reads in the FASTQ file and stored as a uBAM file. The gene sequence of the FASTQ file was compared with the reference genome and the result de-duplicated to obtain a BAM file. The BAM file was combined with the uBAM file to obtain a BAM file with molecular tags. The reads were aggregated and deduplicated according to the molecular tags. The deduplicated reads were used as the input of calling. Calling was to first obtain the original variant set through the pileup method in the panel area, and then filter the blacklist variants. The filtered variant signal was compared with the aforementioned background noise baseline, and the probability of the variant signal coming from the baseline was calculated. If the variant signal was higher than the given threshold, the signal was regarded as background noise. If the variant signal was lower than the given threshold, the signal was regarded as a true variant signal.

The specific method includes the steps of: obtaining variation information of the variant j (Varientⱼ)-VSMj, TSMj, and calling the combined model of the variation according to the coordinates and direction of the variation. The combined model includes the population frequency Pzero at Vaf=0 and the distribution (when vaf≠0). The method further includes the step of performing N times sampling (N=10000) by applying a Monte Carlo Simulation sampling method, generating N×Pzero number of vaf (where vaf=0), generating N×(1−Pzero) number of random vaf based on the variant model of the combined model, and calculating, based on a binomial distribution, the probability Pi of the variant signal (VSMj, TSMj) coming from the noise, wherein each of the N number of vaf is used as a priori noise frequency.

Pi=0, if vafᵢ=0

Pi=1−binomial(n≤VSMⱼ−1|TSMⱼ,vafᵢ) if vafᵢ≠0

The method further includes the step of calculating the summed average of Pi based on the above-mentioned N number of calculation results. The summed average is denoted as P, P=Σ₁^{N}Pi.

The summed average P is used to judge the significance of a single point variation. In the verification, the threshold of the single variation is 0.01. That is, when P≤0.01, the variation is considered to be significantly different from the noise, and is judged as positive; when P≥0.01, the variation is considered to have no significant difference from the noise, and is judged as negative.

1.1.5—Analysis of results—the detection sensitivity of each variant in 3 technical replicates was counted (see Table 1.2), and all the hotspot variants analyzed (including SNV and Indel). The detection sensitivity of hotspot variation with an average vaf of 1% or 0.3% was 100% (where the 95% confidence interval, denoted as CI95, is 90.3%-100%). The detection sensitivity of hotspot variation with an average vaf of 0.1% was 83.3% (CI95, 67.2%-93.6%). The detection sensitivity of hotspot variation with an average vaf of 0.05% was 58.3% (CI95, 40.8%-74.5%). At the same time, it was observed that the detection sensitivities of 12 hotspot variants with similar variant frequencies in the same sample were different, due to the difference in the background noise baseline for each variant.

TABLE 1.2
Sensitivity based on 3 replicate detections for each hotspot
single variant in serially diluted PSC1805 samples

| | PSC1805- | PSC1805- | PSC1805- | PSC1805- | PSC1805-- |
|---|---|---|---|---|---|
| alteration | 1P^{★} | 03P^{⊙} | 01P^{⊙} | 005P^{⊙} | 002P^{⊙} |
| BRAF_V600E | 100.0% | 100.0% | 66.7% | 33.3% | 0.0% |
| EGFR_G719S | 100.0% | 100.0% | 66.7% | 66.7% | 0.0% |
| EGFR_S768I | 100.0% | 100.0% | 100.0% | 100.0% | 0.0% |
| EGFR_T790M | 100.0% | 100.0% | 33.3% | 0.0% | 0.0% |
| EGFR_L858R | 100.0% | 100.0% | 100.0% | 33.3% | 0.0% |
| EGFR_p.E746_A750delELREA | 100.0% | 100.0% | 100.0% | 100.0% | 0.0% |
| KRAS_G12S | 100.0% | 100.0% | 100.0% | 66.7% | 0.0% |
| KRAS_G12D | 100.0% | 100.0% | 66.7% | 0.0% | 0.0% |
| NRAS_G12D | 100.0% | 100.0% | 66.7% | 33.3% | 0.0% |
| NRAS_Q61K | 100.0% | 100.0% | 100.0% | 66.7% | 0.0% |
| NRAS_Q61R | 100.0% | 100.0% | 100.0% | 100.0% | 0.0% |
| PIK3CA_H1047R | 100.0% | 100.0% | 100.0% | 66.7% | 0.0% |
| overall | 100.0% | 100.0% | 83.3% | 58.3% | 0.0% |

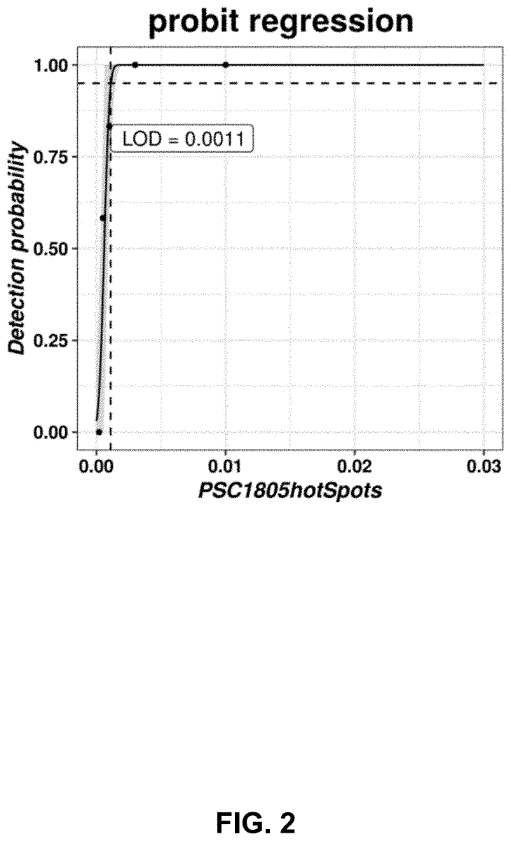

In the standard product, since the coverage depths of these hotspot variants are close and the variation frequencies are similar, a single detection of the 12 variants can be regarded as one variant being detected 12 times. Additionally, since each gradient dilution sample has been performed with 3 repeated experiments, we obtained 36 test results for the variant. We integrated the results of the 36 tests and used the positive detection rate to evaluate the sensitivity of Monte Carlo sampling algorithm based on the combined model for detecting the hotspot variants. Meanwhile, we estimated the minimum detection limit to be 0.11% through Probit regression (FIG. 2).

Specificity analysis of Combined model Monte Carlo sampling algorithm—1.2.1 Sample information—the specificity of Algorithm 1 was evaluated by detecting 19 hotspot-driven variants (listed in Table 1.3) in the plasma samples of 149 healthy people.

TABLE 1.3
List of hotspot-driven variants

| | | | | | COSMIC_ | amidno_acid_ | ddP | nucleotide_ |
|---|---|---|---|---|---|---|---|---|
| Gene | chr | pos | ref | alt | Identifier | change | CR | change |
| KRAS | chr12 | 25398285 | C | T | 517 | G12S | 0.0075 | c.34G > A |
| KRAS | chr12 | 25398281 | C | T | 532 | G13D | ND | c.38G > A |
| KRAS | chr12 | 25378562 | C | T | 19404 | A146T | ND | c.436G > A |
| KRAS | chr12 | 25380276 | T | A | 553 | Q61L | ND | c.182A > T |
| KRAS | chr12 | 25380275 | T | A | 554 | Q61H | ND | c.183A > C |
| KRAS | chr12 | 25398284 | C | T | 521 | G12D | 0.0083 | c.35G > A |
| NRAS | chr1 | 1.15E+08 | C | T | 573 | G13D | 0.0057 | c.38G > A |
| NRAS | chr1 | 1.15E+08 | C | T | 564 | G12D | 0.0072 | c.35G > A |
| NRAS | chr1 | 1.15E+08 | G | T | 580 | Q61K | 0.0076 | c.181C > A |
| NRAS | chr1 | 1.15E+08 | T | C | 584 | Q61R | 0.008 | c.182A > G |
| PIK3CA | chr3 | 1.79E+08 | G | A | 763 | E545K | ND | c.1633G > A |
| PIK3CA | chr3 | 1.79E+08 | G | A | 760 | E542K | ND | c.1624G > A |
| PIK3CA | chr3 | 1.79E+08 | A | G | 775 | H1047R | 0.0089 | c.3140A > G |
| BRAF | chr7 | 1.4E+08 | A | T | 475 | V600E | 0.0092 | c.1799T > A |
| EGFR | chr7 | 55241707 | G | A | 6252 | G719S | 0.0094 | c.2155G > A |
| EGFR | chr7 | 55249005 | G | T | 6241 | S768I | 0.0137 | c.2303G > T |
| EGFR | chr7 | 55249071 | C | T | 6240 | T790M | 0.0088 | c.2369C > T |
| EGFR | chr7 | 55259515 | T | G | 6224 | L858R | 0.0111 | c.2573T > G |
| EGFR | chr7 | 55242464 | AG | A | 6223 | p.E746_A750 | 0.0153 | c.2235_22 |
| | | | GA | | | delELREA | | 49del15 |
| | | | AT | | | | | |
| | | | TA | | | | | |
| | | | AG | | | | | |
| | | | AG | | | | | |
| | | | AA | | | | | |
| | | | GC | | | | | |

1.2.2 Experimental procedure—First, 149 healthy people's plasma samples were extracted with cfDNA by using MagMAX Cell-Free DNA (cfDNA) Isolation. The library construction process, capture process, computer process, and computer data volume are consistent with the aforementioned sensitivity verification experiment process.

1.2.3 Bioinformation analysis was the same as 1.1.4 above.

In this verification, a total of 149×19=2831 detections of variants were performed. The 2831 detection results were all negative. Therefore, the detection specificity of the Monte Carlo sampling algorithm based on the combination model for the hotspot single variation, is 100% (CI95,99.86%-100%).

### Example 5—Performance Analysis of Single Variant Detection Based on Three Algorithms of Combined Model Expected Value, Combined Model Monte Carlo Sampling and MLE

In this embodiment, by analyzing the experimental data for performance verification, the detection sensitivity and specificity of the three analysis procedures for non-hotspot single variants were verified based on three different algorithms. The KAPA Hyper Preparation Kit was used to construct the library, and then PanelP2 was used (Attached Table 6) to enrich the target region. PanelP2 covered a 2.1Mb interval of 769 genes. The enriched library was sequenced with high depth. In the performance evaluation, the sample used was a mixture of the white blood cell DNA of an individual S with known SNP site information and a negative control standard GM12878.

2.1 Sample information—The 32 SNP variants different from hg19 and GM12878 in an individual S were included in a positive variant set (Table 2.1) for sensitivity analysis of three algorithms for detection of the non-hotspot single variants. The 454 SNP loci in the white blood cell DNA of individual S and DNA of cell line GM12878, that have the same genotype as the reference genome hg19, were included in a negative variant set (Table 2.2) for specificity analysis of the three algorithms for detection of the non-hotspot single variants. Specifically, the leukocyte DNA of individual S was serially diluted with normal diploid cell line GM12878 to obtain a series of MAVC2006 samples that can be used for overall performance verification analysis. The series of MAVC2006 samples included 5 dilution gradients, and the expected variation frequencies (vaf) from high to low were 0.5%, 0.3%, 0.1%, 0.05%, and 0.03%, respectively.

TABLE 2.1
SNP information of positive variant set for MAVC2006 samples
SNP information of Positive variant set

| # | chr | pos_raw | ref | alt | gene |
|---|---|---|---|---|---|
| 1 | chr10 | 43610119 | G | A | RET |
| 2 | chr14 | 1.05E+08 | C | T | AKT1 |
| 3 | chr15 | 66729250 | C | T | MAP2K1 |
| 4 | chr16 | 3656625 | G | A | SLX4 |
| 5 | chr17 | 29653293 | T | C | NF1 |
| 6 | chr17 | 29679246 | G | A | NF1 |
| 7 | chr17 | 41246481 | T | C | BRCA1 |
| 8 | chr17 | 56435080 | G | C | RNF43 |
| 9 | chr19 | 2228827 | C | T | DOT1L |
| 10 | chr19 | 5210622 | G | A | PTPRS |
| 11 | chr2 | 2.09E+08 | G | C | IDH1 |
| 12 | chr2 | 29462520 | G | A | ALK |
| 13 | chr21 | 36259181 | T | C | RUNX1 |
| 14 | chr21 | 36262014 | T | A | RUNX1 |
| 15 | chr4 | 1806629 | C | T | FGFR3 |
| 16 | chr4 | 1.88E+08 | T | G | FAT1 |
| 17 | chr4 | 1947324 | G | T | WHSC1 |
| 18 | chr4 | 55129831 | C | T | PDGFRA |
| 19 | chr6 | 1.18E+08 | G | C | ROS1 |
| 20 | chr6 | 1.18E+08 | T | G | ROS1 |
| 21 | chr6 | 1.18E+08 | C | T | ROS1 |
| 22 | chr6 | 1.18E+08 | C | A | ROS1 |
| 23 | chr6 | 1.18E+08 | G | A | ROS1 |
| 24 | chr7 | 2959067 | C | T | CARD11 |
| 25 | chr7 | 55214443 | G | A | EGFR |
| 26 | chr7 | 55248952 | G | A | EGFR |
| 27 | chr9 | 87488402 | C | A | NTRK2 |
| 28 | chr9 | 87488718 | A | G | NTRK2 |
| 29 | chr9 | 87489785 | G | C | NTRK2 |
| 30 | chr9 | 87490546 | C | G | NTRK2 |
| 31 | chr9 | 87491480 | A | C | NTRK2 |
| 32 | chrX | 47424615 | C | T | ARAF |

TABLE 2.2
SNP information of negative variant set for MAVC2006
samples SNP loci information of negative variant set

| # | chrom | pos | ref |
|---|---|---|---|
| 1 | chr1 | 11182192 | C |
| 2 | chr1 | 11199518 | T |
| 3 | chr1 | 11273418 | T |
| 4 | chr1 | 11273640 | G |
| 5 | chr1 | 11303146 | G |
| 6 | chr1 | 11303383 | T |
| 7 | chr1 | 118165648 | A |
| 8 | chr1 | 120466467 | A |
| 9 | chr1 | 120496301 | G |
| 10 | chr1 | 120594140 | G |
| 11 | chr1 | 161332346 | C |
| 12 | chr1 | 16174658 | A |
| 13 | chr1 | 16202813 | G |
| 14 | chr1 | 16254686 | C |
| 15 | chr1 | 16258907 | G |
| 16 | chr1 | 16260309 | C |
| 17 | chr1 | 162746170 | C |
| 18 | chr1 | 17371223 | C |
| 19 | chr1 | 176176119 | A |
| 20 | chr1 | 186007997 | G |
| 21 | chr1 | 186077734 | A |
| 22 | chr1 | 186083224 | G |
| 23 | chr1 | 186107069 | T |
| 24 | chr1 | 186134246 | A |
| 25 | chr1 | 186141181 | C |
| 26 | chr1 | 206648193 | C |
| 27 | chr1 | 226553720 | T |
| 28 | chr1 | 226566838 | C |
| 29 | chr1 | 241661240 | G |
| 30 | chr1 | 241683077 | C |
| 31 | chr1 | 2490631 | T |
| 32 | chr1 | 27023716 | G |
| 33 | chr1 | 43805240 | A |
| 34 | chr1 | 43812255 | A |
| 35 | chr1 | 43812411 | A |
| 36 | chr1 | 45797797 | C |
| 37 | chr1 | 45798260 | T |
| 38 | chr1 | 45800167 | G |
| 39 | chr1 | 45805880 | G |
| 40 | chr1 | 46512289 | T |
| 41 | chr1 | 46597668 | A |
| 42 | chr1 | 46739464 | C |
| 43 | chr1 | 59248806 | C |
| 44 | chr1 | 78415018 | A |
| 45 | chr1 | 78429408 | G |
| 46 | chr1 | 9775972 | T |
| 47 | chr1 | 9780598 | T |
| 48 | chr1 | 9782261 | T |
| 49 | chr1 | 98165122 | T |
| 50 | chr10 | 104268877 | G |
| 51 | chr10 | 104375002 | C |
| 52 | chr10 | 104379249 | T |
| 53 | chr10 | 104913477 | G |
| 54 | chr10 | 123245074 | T |
| 55 | chr10 | 123247644 | A |
| 56 | chr10 | 123325272 | G |
| 57 | chr10 | 123353315 | C |
| 58 | chr10 | 63808960 | T |
| 59 | chr10 | 63851643 | G |
| 60 | chr10 | 70432644 | T |
| 61 | chr1l | 100999633 | C |
| 62 | chr11 | 108098576 | C |
| 63 | chr11 | 108160350 | C |
| 64 | chr11 | 108168053 | A |
| 65 | chr11 | 118307454 | G |
| 66 | chr11 | 118360980 | A |
| 67 | chr11 | 118373677 | C |
| 68 | chr11 | 119170339 | C |
| 69 | chr11 | 119170530 | G |
| 70 | chr11 | 125502486 | A |
| 71 | chr11 | 2154356 | C |
| 72 | chr11 | 2161530 | C |
| 73 | chr11 | 22647274 | G |
| 74 | chr11 | 61204409 | C |
| 75 | chr11 | 85989043 | T |
| 76 | chr11 | 94169053 | C |
| 77 | chr12 | 12022766 | G |
| 78 | chr12 | 12871056 | C |
| 79 | chr12 | 133201467 | C |
| 80 | chr12 | 133209447 | G |
| 81 | chr12 | 133219989 | A |
| 82 | chr12 | 133233901 | G |
| 83 | chr12 | 133254100 | T |
| 84 | chr12 | 133256151 | G |
| 85 | chr12 | 18439811 | G |
| 86 | chr12 | 18747437 | G |
| 87 | chr12 | 25362536 | G |
| 88 | chr12 | 46123647 | C |
| 89 | chr12 | 46123892 | G |
| 90 | chr12 | 46244334 | G |
| 91 | chr12 | 46285551 | T |
| 92 | chr12 | 49421772 | G |
| 93 | chr12 | 49426171 | C |
| 94 | chr12 | 49427347 | C |
| 95 | chr12 | 49445725 | T |
| 96 | chr12 | 49446879 | C |
| 97 | chr12 | 49448792 | A |
| 98 | chr12 | 498088 | G |
| 99 | chr12 | 56479243 | C |
| 100 | chr12 | 56481334 | C |
| 101 | chr12 | 56492352 | G |
| 102 | chr12 | 69202729 | T |
| 103 | chr12 | 69222593 | G |
| 104 | chr13 | 28674595 | G |
| 105 | chr13 | 28908288 | G |
| 106 | chr13 | 28960084 | G |
| 107 | chr13 | 28960566 | A |
| 108 | chr13 | 28962942 | C |
| 109 | chr13 | 32906480 | A |
| 110 | chr13 | 32906902 | A |
| 111 | chr13 | 32910614 | T |
| 112 | chr13 | 32912928 | G |
| 113 | chr13 | 32914277 | A |
| 114 | chr13 | 32929478 | C |
| 115 | chr13 | 32945123 | A |
| 116 | chr13 | 73349527 | C |
| 117 | chr13 | 73350235 | G |
| 118 | chr14 | 105238820 | G |
| 119 | chr14 | 105241255 | C |
| 120 | chr14 | 105246407 | G |
| 121 | chr14 | 105259034 | G |
| 122 | chr14 | 20822219 | G |
| 123 | chr14 | 65542071 | T |
| 124 | chr14 | 68944357 | T |
| 125 | chr14 | 69028855 | T |
| 126 | chr14 | 69029996 | C |
| 127 | chr14 | 69030263 | C |
| 128 | chr14 | 69061753 | G |
| 129 | chr14 | 75485519 | G |
| 130 | chr14 | 75489531 | G |
| 131 | chr14 | 75497239 | G |
| 132 | chr14 | 75513534 | G |
| 133 | chr14 | 81606063 | G |
| 134 | chr14 | 95560205 | T |
| 135 | chr14 | 95582861 | T |
| 136 | chr15 | 41021696 | C |
| 137 | chr15 | 66679684 | A |
| 138 | chr15 | 66774267 | G |
| 139 | chr15 | 67418336 | T |
| 140 | chr15 | 88524609 | C |
| 141 | chr15 | 88679689 | G |
| 142 | chr15 | 91312405 | T |
| 143 | chr15 | 91333894 | A |
| 144 | chr15 | 99442891 | A |
| 145 | chr15 | 99465343 | G |
| 146 | chr15 | 99467189 | A |
| 147 | chr16 | 14015921 | G |
| 148 | chr16 | 2097879 | T |
| 149 | chr16 | 2108755 | A |
| 150 | chr16 | 2125788 | C |
| 151 | chr16 | 2129454 | C |
| 152 | chr16 | 2134572 | C |
| 153 | chr16 | 2138218 | A |
| 154 | chr16 | 2223851 | C |
| 155 | chr16 | 347044 | C |
| 156 | chr16 | 349240 | G |
| 157 | chr16 | 3843587 | G |
| 158 | chr16 | 67671804 | T |
| 159 | chr16 | 68849613 | A |
| 160 | chr16 | 68856080 | C |
| 161 | chr16 | 81904471 | C |
| 162 | chr16 | 81914493 | T |
| 163 | chr16 | 81965072 | T |
| 164 | chr16 | 81969647 | C |
| 165 | chr16 | 89805210 | C |
| 166 | chr16 | 89865003 | C |
| 167 | chr16 | 89865225 | C |
| 168 | chr17 | 15965268 | G |
| 169 | chr17 | 15965400 | A |
| 170 | chr17 | 17119838 | C |
| 171 | chr17 | 29562582 | A |
| 172 | chr17 | 29587341 | G |
| 173 | chr17 | 30264366 | C |
| 174 | chr17 | 33428357 | C |
| 175 | chr17 | 37884233 | G |
| 176 | chr17 | 40485682 | A |
| 177 | chr17 | 41201105 | T |
| 178 | chr17 | 41244838 | C |
| 179 | chr17 | 41244982 | A |
| 180 | chr17 | 41245067 | T |
| 181 | chr17 | 56435243 | T |
| 182 | chr17 | 62009538 | C |
| 183 | chr17 | 63531768 | G |
| 184 | chr17 | 63533087 | C |
| 185 | chr17 | 70120551 | A |
| 186 | chr17 | 78858769 | C |
| 187 | chr17 | 7978880 | T |
| 188 | chr18 | 39617631 | T |
| 189 | chr18 | 60970074 | G |
| 190 | chr19 | 10291181 | T |
| 191 | chr19 | 11097111 | A |
| 192 | chr19 | 11097696 | A |
| 193 | chr19 | 1222974 | G |
| 194 | chr19 | 1223997 | G |
| 195 | chr19 | 1225052 | G |
| 196 | chr19 | 1226083 | G |
| 197 | chr19 | 15281459 | C |
| 198 | chr19 | 15303381 | A |
| 199 | chr19 | 15383888 | C |
| 200 | chr19 | 17945569 | T |
| 201 | chr19 | 17946702 | T |
| 202 | chr19 | 17952532 | T |
| 203 | chr19 | 18273330 | C |
| 204 | chr19 | 18279640 | G |
| 205 | chr19 | 2210606 | C |
| 206 | chr19 | 2211146 | T |
| 207 | chr19 | 2216592 | G |
| 208 | chr19 | 2229045 | A |
| 209 | chr19 | 30308274 | C |
| 210 | chr19 | 40741070 | G |
| 211 | chr19 | 4101320 | G |
| 212 | chr19 | 4102820 | G |
| 213 | chr19 | 41727769 | C |
| 214 | chr19 | 42797228 | C |
| 215 | chr19 | 42797682 | C |
| 216 | chr19 | 45855705 | G |
| 217 | chr19 | 45867824 | G |
| 218 | chr19 | 45868291 | T |
| 219 | chr19 | 5260765 | G |
| 220 | chr19 | 5260797 | T |
| 221 | chr19 | 52725338 | T |
| 222 | chr19 | 5286171 | T |
| 223 | chr19 | 55452849 | C |
| 224 | chr2 | 128051309 | C |
| 225 | chr2 | 178128179 | C |
| 226 | chr2 | 178128362 | C |
| 227 | chr2 | 198273243 | T |
| 228 | chr2 | 198283600 | T |
| 229 | chr2 | 202131347 | G |
| 230 | chr2 | 209108226 | T |
| 231 | chr2 | 212286797 | A |
| 232 | chr2 | 212426708 | A |
| 233 | chr2 | 215645609 | C |
| 234 | chr2 | 216212339 | T |
| 235 | chr2 | 223083542 | G |
| 236 | chr2 | 242801011 | A |
| 237 | chr2 | 26022399 | A |
| 238 | chr2 | 26101006 | G |
| 239 | chr2 | 47602405 | G |
| 240 | chr2 | 47637371 | A |
| 241 | chr2 | 47710098 | G |
| 242 | chr2 | 61722778 | G |
| 243 | chr2 | 61753510 | C |
| 244 | chr2 | 68400639 | G |
| 245 | chr2 | 96920526 | C |
| 246 | chr2 | 99182262 | A |
| 247 | chr20 | 30946706 | G |
| 248 | chr20 | 31375014 | C |
| 249 | chr20 | 31383160 | A |
| 250 | chr20 | 31384607 | T |
| 251 | chr20 | 36024591 | T |
| 252 | chr20 | 39658155 | C |
| 253 | chr20 | 40710573 | G |
| 254 | chr20 | 40730751 | G |
| 255 | chr20 | 40877308 | G |
| 256 | chr20 | 44756908 | A |
| 257 | chr20 | 49354288 | T |
| 258 | chr20 | 54945383 | A |
| 259 | chr20 | 57428199 | C |
| 260 | chr20 | 57429696 | C |
| 261 | chr21 | 36164479 | T |
| 262 | chr21 | 36206730 | G |
| 263 | chr21 | 36261011 | G |
| 264 | chr21 | 39751929 | G |
| 265 | chr21 | 39764304 | A |
| 266 | chr21 | 42866388 | A |
| 267 | chr21 | 45646899 | A |
| 268 | chr21 | 45648905 | G |
| 269 | chr22 | 21272210 | C |
| 270 | chr22 | 24143308 | C |
| 271 | chr22 | 32211339 | C |
| 272 | chr22 | 32211416 | A |
| 273 | chr22 | 41513285 | G |
| 274 | chr22 | 41523770 | G |
| 275 | chr22 | 41543949 | C |
| 276 | chr22 | 41564718 | T |
| 277 | chr3 | 10070336 | G |
| 278 | chr3 | 10128901 | T |
| 279 | chr3 | 10141042 | C |
| 280 | chr3 | 10183876 | G |
| 281 | chr3 | 10191719 | C |
| 282 | chr3 | 119545628 | G |
| 283 | chr3 | 12393125 | C |
| 284 | chr3 | 12422809 | C |
| 285 | chr3 | 124456742 | G |
| 286 | chr3 | 12639419 | A |
| 287 | chr3 | 12639596 | C |
| 288 | chr3 | 134670908 | C |
| 289 | chr3 | 134920306 | C |
| 290 | chr3 | 138474791 | T |
| 291 | chr3 | 142171199 | c |
| 292 | chr3 | 142277595 | T |
| 293 | chr3 | 187451313 | T |
| 294 | chr3 | 189349083 | T |
| 295 | chr3 | 189349175 | C |
| 296 | chr3 | 189526354 | T |
| 297 | chr3 | 37067240 | T |
| 298 | chr3 | 41268671 | A |
| 299 | chr3 | 41274815 | C |
| 300 | chr3 | 47158087 | A |
| 301 | chr3 | 47165219 | T |
| 302 | chr3 | 47165872 | T |
| 303 | chr3 | 47205320 | G |
| 304 | chr3 | 51978529 | C |
| 305 | chr3 | 52440418 | A |
| 306 | chr3 | 69987775 | C |
| 307 | chr3 | 71021303 | T |
| 308 | chr3 | 72864491 | G |
| 309 | chr3 | 89448991 | A |
| 310 | chr4 | 106157703 | T |
| 311 | chr4 | 106158738 | G |
| 312 | chr4 | 106158795 | A |
| 313 | chr4 | 106162344 | C |
| 314 | chr4 | 106194010 | A |
| 315 | chr4 | 106194083 | T |
| 316 | chr4 | 106196405 | C |
| 317 | chr4 | 106196829 | T |
| 318 | chr4 | 153332301 | C |
| 319 | chr4 | 17666416 | C |
| 320 | chr4 | 1803329 | G |
| 321 | chr4 | 183650006 | C |
| 322 | chr4 | 187509861 | G |
| 323 | chr4 | 187539588 | T |
| 324 | chr4 | 187540683 | A |
| 325 | chr4 | 1932537 | A |
| 326 | chr4 | 1943549 | A |
| 327 | chr4 | 3210510 | C |
| 328 | chr4 | 55968623 | A |
| 329 | chr4 | 66196635 | G |
| 330 | chr4 | 66201669 | G |
| 331 | chr4 | 66231683 | A |
| 332 | chr4 | 84405190 | T |
| 333 | chr5 | 112043384 | T |
| 334 | chr5 | 112043620 | G |
| 335 | chr5 | 112116587 | A |
| 336 | chr5 | 112128212 | G |
| 337 | chr5 | 118532118 | A |
| 338 | chr5 | 1268624 | G |
| 339 | chr5 | 142421382 | G |
| 340 | chr5 | 149433857 | C |
| 341 | chr5 | 149435946 | A |
| 342 | chr5 | 149439458 | T |
| 343 | chr5 | 149457015 | T |
| 344 | chr5 | 149460617 | G |
| 345 | chr5 | 170221307 | G |
| 346 | chr5 | 170832369 | G |
| 347 | chr5 | 176637243 | T |
| 348 | chr5 | 176638695 | A |
| 349 | chr5 | 180057293 | T |
| 350 | chr5 | 223646 | A |
| 351 | chr5 | 231143 | T |
| 352 | chr5 | 236536 | T |
| 353 | chr5 | 254599 | A |
| 354 | chr5 | 35873571 | C |
| 355 | chr5 | 38955694 | C |
| 356 | chr5 | 39074377 | T |
| 357 | chr5 | 56116303 | A |
| 358 | chr5 | 56116534 | C |
| 359 | chr5 | 67584357 | A |
| 360 | chr5 | 79951491 | T |
| 361 | chr5 | 79952348 | C |
| 362 | chr5 | 86564492 | G |
| 363 | chr5 | 86679519 | C |
| 364 | chr6 | 106546506 | T |
| 365 | chr6 | 106547372 | C |
| 366 | chr6 | 106555334 | A |
| 367 | chr6 | 117642418 | A |
| 368 | chr6 | 117650532 | C |
| 369 | chr6 | 117650563 | A |
| 370 | chr6 | 117677875 | T |
| 371 | chr6 | 117717348 | T |
| 372 | chr6 | 138196066 | T |
| 373 | chr6 | 138200114 | A |
| 374 | chr6 | 142691874 | A |
| 375 | chr6 | 157150568 | C |
| 376 | chr6 | 157405967 | C |
| 377 | chr6 | 157488357 | C |
| 378 | chr6 | 157511267 | A |
| 379 | chr6 | 162137147 | C |
| 380 | chr6 | 162864338 | T |
| 381 | chr6 | 20490390 | T |
| 382 | chr6 | 26032306 | G |
| 383 | chr6 | 26056085 | T |
| 384 | chr6 | 76728475 | G |
| 385 | chr6 | 94120639 | T |
| 386 | chr7 | 116339770 | T |
| 387 | chr7 | 116371946 | C |
| 388 | chr7 | 128845188 | C |
| 389 | chr7 | 13948287 | G |
| 390 | chr7 | 13995882 | T |
| 391 | chr7 | 140419863 | C |
| 392 | chr7 | 140423507 | C |
| 393 | chr7 | 140424582 | G |
| 394 | chr7 | 140425887 | C |
| 395 | chr7 | 148511048 | C |
| 396 | chr7 | 151846108 | G |
| 397 | chr7 | 151846114 | A |
| 398 | chr7 | 151853327 | T |
| 399 | chr7 | 151877227 | C |
| 400 | chr7 | 151949694 | A |
| 401 | chr7 | 2962201 | A |
| 402 | chr7 | 2972204 | G |
| 403 | chr7 | 2978310 | C |
| 404 | chr7 | 2987193 | G |
| 405 | chr7 | 50800201 | T |
| 406 | chr7 | 55229165 | C |
| 407 | chr7 | 6026864 | G |
| 408 | chr7 | 6414414 | C |
| 409 | chr7 | 6414442 | G |
| 410 | chr8 | 145741388 | C |
| 411 | chr8 | 55371903 | A |
| 412 | chr8 | 56879470 | A |
| 413 | chr8 | 68972907 | C |
| 414 | chr8 | 69017721 | C |
| 415 | chr9 | 101585531 | T |
| 416 | chr9 | 101589100 | A |
| 417 | chr9 | 101602476 | G |
| 418 | chr9 | 101910087 | T |
| 419 | chr9 | 110250491 | G |
| 420 | chr9 | 133738395 | C |
| 421 | chr9 | 135772614 | G |
| 422 | chr9 | 135782221 | T |
| 423 | chr9 | 135782769 | A |
| 424 | chr9 | 135786112 | T |
| 425 | chr9 | 135797176 | G |
| 426 | chr9 | 21991652 | T |
| 427 | chr9 | 37026702 | G |
| 428 | chr9 | 40500077 | T |
| 429 | chr9 | 5522617 | G |
| 430 | chr9 | 8338878 | A |
| 431 | chr9 | 8376601 | G |
| 432 | chr9 | 8633487 | G |
| 433 | chr9 | 87428029 | A |
| 434 | chr9 | 87487388 | G |
| 435 | chr9 | 87487610 | A |
| 436 | chr9 | 87488521 | G |
| 437 | chr9 | 87488593 | C |
| 438 | chr9 | 87489848 | C |
| 439 | chr9 | 87563370 | T |
| 440 | chr9 | 97872748 | C |
| 441 | chr9 | 97872834 | T |
| 442 | chr9 | 97873435 | G |
| 443 | chr9 | 98211297 | G |
| 444 | chr9 | 98240437 | G |
| 445 | chrX | 100617567 | A |
| 446 | chrX | 118215351 | A |
| 447 | chrX | 153176655 | G |
| 448 | chrX | 44966795 | T |
| 449 | chrX | 47041734 | C |
| 450 | chrX | 47430769 | G |
| 451 | chrX | 63406128 | G |
| 452 | chrX | 63407623 | A |
| 453 | chrX | 76856039 | C |
| 454 | chrX | 76871649 | C |

2.2 Experiential procedure—The five series of MAVC2006 samples were fragmented using Covaris. By taking into account the influence of the initial amount of library construction on the sensitivity of detection, the sensitivity and specificity was evaluated of single variant detection with the initial amount of 5 ng, 15 ng, 40 ng and 100 ng for DNA library construction, respectively. KAPA Hyper Preparation Kit was used for library construction, PanelP2 was used for target area capture, and Novaseq was used for sequencing, with an average sequencing depth of 7300×.

2.3 PanelP2 baseline model construction—2.3.1 Baseline model construction based on combined model (expected value/Monte Carlo sampling) algorithm.

The construction of the baseline model was based on the plasma free DNA data of 2000 negative populations. The experimental procedures such as the construction, capture, and computerization of the plasma library and the data volume on the computer were completely consistent with the aforementioned standard products. Before constructing the model, the subtraction of germline mutations and clonal hematopoietic mutations was first performed. In particular, when the data came from tumor patients, tumor tissue-specific mutations were also subtracted. Then, outlier processing to reduce noise was performed. The remaining variation represented the noise signal of each variation direction (Subtype) of each chromosome coordinate (Position). In this example, the combined model was used to fit the baseline noise signal model, record the proportion of non-variant populations corresponding to each variation direction (Subtype) of each chromosome coordinate (Position), perform Weibull distribution simulation on the vaf of the variant population, and calculate the expected value of the fitted model.

2.3.2 Baseline model construction based on MLE algorithm—the same batch of samples were used as 2.3.1 to build the baseline model of the MLE algorithm. Similarly, before the model was built, subtraction of germline mutations and clonal hematopoietic mutations was performed. Particularly, when the data came from tumor patients, the tumor tissue-specific mutations were also subtracted. Then, outlier processing was performed to reduce noise. The remaining variation represented the noise signal of each variation direction (Subtype) of each chromosome coordinate (Position). In this embodiment, a single model (binomial model, that is, algorithm 2) was used to fit the baseline signal model, and use the noise data of the baseline population through a likelihood function to fit the distribution of the occurrence probability θnoise of the plasma noise signal (VSM, TSM) for a specific variation at a specific locus. The distribution of the occurrence probability θnoise is denoted as f(θnoise). The likelihood function is, L(f(θnoise)|VSM,TSM)=Π₁ⁿbinomial (VSMi, TSMi, f(θnoise)).

2.4 Bioinformation analysis—The gene sequence of the FASTQ file was compared with the reference genome and deduplicated to obtain a BAM file. The reads were aggregated and deduplicated, and the deduplicated reads were used as the input of calling. Calling is to first obtain the original variant set through the pileup method in the panel area, and then filter the blacklist variants. The filtered variant signal was compared with the above-mentioned background noise baseline, and the probability of the variant different from the baseline was calculated. If the calculated probability was higher than the given threshold, it was considered background noise.

2.4.1 Analysis of algorithm based on combined model expected value—The expected value of the combined model was substituted into the model as a parameter, and the significance of the variation to be measured was calculated. According to the position information of the plasma variation locus, the combined variant model of the locus was called. The vaf expectation of the non-variant population was 0, and the weight was the proportion of the non-variant population to the whole population (Pzero). The vaf expectation value of the variant population was E(P), and its weight was 1−Pzero. Using the expected values of these two models, first the probability of the patient's plasma variation signals (VSMj, TSMj) was calculated from noise signals, and then use the weighted average Pⱼ to measure the significance of the patient's plasma variant signal. The weighted average Pⱼ was calculated by,

Pⱼ=(1−P_{zero})*(1−binomial(n≤VSMⱼ−1|TSMⱼ,E(P))).

The lower the P was, the greater the difference between the baseline noise and the negative population was. In this verification, the single variant significance cutoff was set to be 0.01. That is, when the P value≤0.01, the variant was considered to be significantly different from the noise and judged as positive; when the P value>0.01, the variant was considered to have no significant difference from the noise, Judged as negative.

2.4.2 Analysis of algorithm based on combined model Monte Carlo sampling—Variation information was obtained (VSMj, TSMj) of variation j (Varient j), and called according to the combined model of the variation based on the coordinates and direction of the variation. The combined model includes parameter of population frequency Pzero at vaf=0 and the distribution (at vaf≠0). N times sampling (N=10000) was performed by applying Monte Carlo Simulation sampling method, to generate N×Pzero number of vaf=0, and generate N×(1−Pzero) number of random vaf based on the variant model part. Then each of the N number of vaf was used as a prior noise frequency, respectively, to calculate the probability of the variant signal (VSMj, TSMj) coming from noise according to a binomial distribution. The calculation is expressed by,

Pi=0, if vafᵢ=0

Pi=1−binomial(n≤VSMⱼ−1|TSMⱼ,vafᵢ) if vafᵢ≠0

By combining the N number of calculation results, a summed average of Pi was further calculated. The summed average P was calculated by,

P=Σ₁^{N}Pi

P is a measure of the significance of a single point variation. In this verification, the single variation significance threshold was 0.01. That is, when P≤0.01, the variation was considered to be significantly different from the noise, and was judged as positive; when P≥0.01, the variation was considered to have no significant difference from the noise, and was judged as negative.

2.4.3 Analysis of algorithm based on MLE—Variation information (VSMj, TSMj) of the variation j (Varient j) was obtained, and distribution of the noise signal θnoise was called based on the single model of the variation according to the coordinates and direction of the variation, where the distribution of the noise signal was denoted as f(θnoise). The noise signal distribution f(θnoise) of the variation was substituted in the binomial model, and combined with the VSMj and TSMj of the variation to calculate the significance of the variation in the sample. The single variation significance cutoff was set to be 0.0001. That is, when P≤0.0001, the variation was considered significantly different from noise, and was judged as positive; when P>0.0001, the variation was considered to have no significant difference from the noise, and was judged as negative.

2.5 Analysis of results—The positive variant set of MAVC2006 contained 32 variants. MAVC2006 was diluted with 5 dilution gradients (0.03%, 0.05%, 0.1%, 0.3%, 0.5%). 32×5=160 times of variant detections were integrated to generate statistical results for detection sensitivity. The Table 2.3 shows the detection sensitivity of the three algorithms, respectively. At the same time, the negative variation set of the standard MAVC2006 contained 454 theoretically non-variant loci. 454×5=2270 times of variant detections were also integrated to generate statistical results for detection specificity. The Table 2.3 also shows the detection specificity of the three algorithms. As shown in Table 2.3. The sensitivities of the three algorithms are close, and the sensitivity of the combined model sampling algorithm is the highest. The specificities of the three algorithms can all reach more than 99.7%, and the positive predictive values (PPV) of the three algorithms are all higher than 90%. (NPV is short for negative predictive value).

TABLE 2.3
Overall performance of the three algorithms

| Method | sn | sp | ppv | npv |
|---|---|---|---|---|
| Combined model | 0.46875 | 0.999119 | 0.974026 | 0.963876 |
| expected value | | | | |
| algorithm | | | | |
| Combined model | 0.51875 | 0.997247 | 0.929972 | 0.967105 |
| sampling algorithm | | | | |
| Single model MLE | 0.478125 | 0.999229 | 0.977636 | 0.964495 |
| algorithm | | | | |

### Example 6—Analysis of Sample Detection Performance During Multi-Variant Tracking—Based on Combined Model Monte Carlo Sampling Algorithm

Since the content of cfDNA in the blood limits the sensitivity of single variant detection, the combined model Monte Carlo sampling can be used to track multiple tissue prior tumor-specific variants at the same time to significantly improve the overall detection sensitivity. In the MAVC2006 series of samples, different proportions of mixed DNA were used to simulate plasma DNA with different proportions of tumors. In order to reduce the impact of loci sampling, 100 random samplings were performed by a computer for each designated number of variants, that is, 100 independent priori variant maps of tumors were formed. For each diluted sample, the variant signal of the designated locus was traced according to each of the 100 maps and an MRD status was determined accordingly, therefore, a total of 100 determinations were performed. Finally, the positive detection rates of the 100 samplings were counted as the detection performance of the sample for tracking the designated number of variants.

3.1 Analysis of detection sensitivity for tracking multi-variant based on combined model Monte Carlo sampling—First, a number of variants for tracking were designated, randomly selecting the designated number of variants from the positive variant set, which was a simulation to a priori tumor variation map, specified variants in the sample were tracked, and MRD status of the sample was determined based on the detection. According to the designated number of variants for tracking, 100 random samplings were performed with replacement, each sampling result as a priori variation map, and detection rates of the 100 samplings counted as the detection sensitivity of the sample.

3.1.1 Sample information—In this embodiment, the above-mentioned 5 gradient dilution samples of MAVC2006 were used. A specified number of variants was randomly selected from the 32 variants included in the positive variant set to track, that is, to simulate a priori tumor variant map. The number of variants to track was 1, 2, 3, 6, 10, and 20, to verify the detecting sensitivity of algorithm based on the combined model Monte Carlo sampling.

3.1.2 Experimental procedure—the sensitivity and specificity of single variant detection were evaluated with the initial amount of 5 ng, 15 ng, 40 ng and 100 ng for DNA library construction, respectively. First, the 5 series of MAVC2006 samples were fragmented using Covaris. By taking into account the influence of the initial amount of library construction on the detection sensitivity, the sensitivity of multi-variant detection was evaluated with the initial amount of 15 ng and 40 ng for library construction, respectively. The construction, target area capture and computerization strategy are consistent with the process 2.2, described above

3.1.3 Baseline model construction of algorithm based on combined model Monte Carlo sampling—The same as baseline model construction of 2.3.1, as described above.

3.1.4 Bioinformation analysis—The gene sequence of the FASTQ file was compared with the reference genome and deduplicated to obtain a BAM file. The reads were aggregated and deduplicated, and the deduplicated reads were used as the input of calling. Calling was to first obtain the original variant set through the pileup method in the panel area, and filter the blacklist variant. The filtered variant signal was compared with the above-mentioned background noise baseline, and the probability of the variant different from the baseline was calculated. If the calculated probability of the variant was higher than the given threshold, the variant signal was considered background noise.

Variation information (VSMj, TSMj) was obtained of variation j (Varient j), and called by the combined model of the variation according to the coordinates and direction of the variation. The combined model included a population frequency Pzero at vaf=0 and the distribution (at vaf≠0). N times of sampling (N=10000) was performed by applying Monte Carlo Simulation sampling method. As such, N×Pzero number of vaf=0 were generated, and N×(1−Pzero) number of random vaf were generated based on the variant model part, respectively. N vaf was used as a prior noise frequency, to calculate the probability of the variant signal (VSMj, TSMj) coming from noise according to a binomial distribution. The probability was calculated by,

Pi=0, if vafᵢ=0

Pi=1−binomial(n≤VSMⱼ−1|TSMⱼ,vafᵢ) if vaf≠0

N number of calculation results were combined, and a summed average of Pi was further calculated. The summed average P is expressed by,

P=Σ₁^{N}Pi

The summed average P was a measure of the significance of the single point variation. In this verification, significance threshold of a single variation was defined as cutoff1=0.05. When P≤0.05 for a single variation, the P value of the variation was included in the multi-variant combination analysis; otherwise, the P value of the variation was not included. The MRD sample judgment threshold was defined as cutoff2=0.01. That is, when the P value obtained by multi-variant joint confidence probability analysis was ≤0.01, it was considered that the degree of variation of the sample was significantly different from the noise, and it is judged as MRD+; when P>0.01, the variation of the sample was considered to have no significant difference from the noise, and was judged as MRD−.

3.1.5 Analysis of results—the sample level detection sensitivity of the algorithm based on the combined model Monte Carlo sampling was counted when the number of variants to track was 1, 2, 3, 6, 10, and 20. The detection details are shown in Table 3.1. With an increased initial amount of library construction, and an increased number of variants to track, the detection sensitivity was significantly improved.

TABLE 3.1
Positive detection rates of tracking different numbers of variants.

| | | | Positive detection rates of tracking 1, 2, | | | | | |
|---|---|---|---|---|---|---|---|---|
| Sample information | | | 3, 6, 10 and 20 variants, respectively. | | | | | |
| MAVC-15N-05P | 15 | 0.5 | 100% | 100% | 100% | 100% | 100% | 100% |
| MAVC-15N-03P | 15 | 0.3 | 89% | 99% | 100% | 100% | 100% | 100% |
| MAVC-15N-01P | 15 | 0.1 | 29% | 51% | 64% | 95% | 100% | 100% |
| MAVC-15N-005P | 15 | 0.05 | 21% | 53% | 60% | 93% | 98% | 100% |
| MAVC-15N-003P | 15 | 0.03 | 20% | 35% | 50% | 73% | 94% | 100% |
| MAVC-40N-05P | 40 | 0.5 | 100% | 100% | 100% | 100% | 100% | 100% |
| MAVC-40N-03P | 40 | 0.3 | 100% | 100% | 100% | 100% | 100% | 100% |
| MAVC-40N-01P | 40 | 0.1 | 66% | 86% | 97% | 99% | 100% | 100% |
| MAVC-40N-005P | 40 | 0.05 | 32% | 42% | 65% | 92% | 99% | 100% |
| MAVC-40N-003P | 40 | 0.03 | 15% | 29% | 48% | 70% | 89% | 100% |

3.2 Analysis of detection specificity for tracking multi-variant based on combined model Monte Carlo sampling—First, a number of variants were designated to track, and the designated number of variants were randomly selected from the negative variant set, in order to simulate a priori tumor variation map, track the specified variants in the sample, and determine the MRD status of the sample based on the detection. According to the designated number of variants for tracking, 100 random samplings with replacement were performed, each sampling resulted in an a priori variation map, and the detection rates of the 100 samplings counted as a false positive rate at a sample level, and thereafter used to calculate the detection specificity.

3.2.1 Sample information—This example used the above-mentioned five series of MAVC2006 samples. The negative variant set contained 454 homozygous SNP loci, and the genotypes of these loci were consistent with the reference genome hg19. Taking into account the influence of the initial amount of library construction on the detection sensitivity, the influence of the initial amounts of 5 ng, 15 ng, 40 ng and 100 ng were evaluated on the sensitivity of multi- variant detection, respectively. In this embodiment, detection specificity was evaluated for the algorithm based on combined model Monte Carlo sampling when the numbers of variants to track were 2, 3, 6, 10, 20, 50, and 100.

3.2.1 Experimental procedure—The same procedure as 3.1.2 above was used.

3.2.3 Bioinformation analysis—The same procedure as 3.1.4 above was used.

3.2.4 Analysis of results—The detection status was counted of loci based on combined model Monte Carlo sampling when the numbers of variants to track were 1, 2, 3, 6, 10, 20, 50, and 100. The detection rate details are shown in Table 3.2. When tracking different numbers of variants, the specificity of the detections was steadily maintained between 99.7%-99.9%, and the specificity was not decreased due to track of more loci.

TABLE 3.2
Detection specificity of tracking different numbers of variants in the negative variant set.

| | | | False positive rate of tracking different numbers of variants in the | | | | | | | |
|---|---|---|---|---|---|---|---|---|---|---|
| Sample Information | | | negative variant set | | | | | | | |
| SAMPLE Name | input(ng) | VAF(%) | 1 | 2 | 3 | 6 | 10 | 20 | 50 | 100 |
| MAVC-5N-05P | 5 | 0.5 | 0% | 0% | 0% | 0% | 0% | 0% | 0% | 0% |
| MAVC-5N-03P | 5 | 0.3 | 0% | 0% | 0% | 0% | 0% | 0% | 0% | 0% |
| MAVC-5N-01P | 5 | 0.1 | 1% | 0% | 0% | 0% | 0% | 0% | 0% | 0% |
| MAVC-5N-005P | 5 | 0.05 | 0% | 1% | 1% | 2% | 0% | 0% | 0% | 0% |
| MAVC-5N-003P | 5 | 0.03 | 0% | 0% | 0% | 0% | 0% | 0% | 0% | 0% |
| MAVC-15N-05P | 15 | 0.5 | 0% | 0% | 0% | 0% | 0% | 0% | 0% | 0% |
| MAVC-15N-03P | 15 | 0.3 | 0% | 0% | 0% | 0% | 0% | 0% | 0% | 0% |
| MAVC-15N-01P | 15 | 0.1 | 0% | 0% | 0% | 0% | 0% | 0% | 0% | 0% |
| MAVC-15N-005P | 15 | 0.05 | 0% | 0% | 0% | 0% | 0% | 0% | 0% | 0% |
| MAVC-15N-003P | 15 | 0.03 | 1% | 0% | 0% | 0% | 1% | 0% | 0% | 0% |
| MAVC-40N-05P | 40 | 0.5 | 0% | 0% | 0% | 0% | 0% | 0% | 0% | 0% |
| MAVC-40N-03P | 40 | 0.3 | 0% | 0% | 0% | 1% | 0% | 1% | 1% | 0% |
| MAVC-40N-01P | 40 | 0.1 | 1% | 0% | 1% | 1% | 2% | 2% | 2% | 0% |
| MAVC-40N-005P | 40 | 0.05 | 0% | 0% | 0% | 0% | 0% | 0% | 0% | 0% |
| MAVC-40N-003P | 40 | 0.03 | 0% | 0% | 0% | 0% | 0% | 1% | 1% | 0% |
| MAVC-100N-05P | 100 | 0.5 | 0% | 0% | 0% | 0% | 0% | 0% | 0% | 0% |
| MAVC-100N-03P | 100 | 0.3 | 0% | 0% | 0% | 0% | 0% | 0% | 0% | 0% |
| MAVC-100N-01P | 100 | 0.1 | 0% | 0% | 0% | 0% | 0% | 0% | 0% | 0% |
| MAVC-100N-005P | 100 | 0.05 | 0% | 0% | 0% | 0% | 0% | 0% | 0% | 0% |
| MAVC-100N-003P | 100 | 0.03 | 2% | 0% | 1% | 2% | 1% | 0% | 0% | 0% |
| Specificity (overall) | | | 99.75% | 99.95% | 99.85% | 99.70% | 99.80% | 99.80% | 99.80% | 99.75% |

### Example 7—4 Performance Analysis of MRD Detection in Lung Cancer Cohort Based on Combined Model Monte Carlo Sampling Algorithm

This embodiment used a tissue priori strategy to perform MRD detection on plasma samples of 27 patients with non-small cell lung cancer at different time points, which was combined with the actual clinical relapse of the patient, to verify the clinical performance of the technology and the algorithm. In this small cohort study, the median follow-up time of patients reached 505 days (166-870 days), of which 14 patients relapsed and 13 did not relapse. In this test, a fixed PanelP3 (attached table 7) was used covering the 2.4Mb region of 1631 genes to enrich the target region.

4.1 Patient information and sample information—This case covers 27 patients with non-small cell lung cancer with tumor stages from stage I to stage III, including 7 cases in stage I, 14 cases in stage II, and 6 cases in stage III (see Table 3.1 for details). All of the patients have undergone radical surgical treatment and were collected with intraoperative tissue samples. During the 30-month follow-ups of these patients, blood samples were collected at multiple time points, including 3 days after surgery, 2 weeks after surgery, and one month after surgery, etc.

4.2 Experimental procedure—The collected intraoperative tissue samples and albuginea were extracted using the “Tiangen Blood/Tissue/Cell Genome Extraction Kit”. The plasma samples were extracted using MagMAX Cell-Free DNA (cfDNA) Isolation for cell-free DNA extraction. For all three types of DNA samples, KAPA Hyper Preparation Kit was used for library construction. PanelP3 was used for target area capture of tissue, white blood cell samples and plasma cfDNA. The average sequencing depth of plasma cell-free DNA library was about 8700×, and the average sequencing depth of tissue and white blood cell genomic DNA was 1000×. First, the tissues and paired BCs were sequenced to establish a patient's tumor-specific variant map. Then the variant in the map was specifically tracked in the blood, and the MRD status of the sample was determined based on the combined model Monte Carlo sampling algorithm.

4.3 PanelP3 baseline model construction: The construction of the baseline model was based on the plasma free DNA data of 1837 negative people. The construction, capture, and computer operation of the plasma library and the amount of data on the computer were completely consistent with the aforementioned experimental procedure of patient plasma (4.2). Before constructing the model, the subtraction of germline mutations and clonal hematopoietic mutations was first performed. In particular, when the data came from tumor patients, tumor tissue-specific mutations were also subtracted. Then, outlier processing was performed to reduce noise, and the remaining variation represented the noise signal of each variation direction (Subtype) of each chromosome coordinate (Position). In this example, the combined model was used to fit the baseline noise signal model, record the proportion of non-variant population corresponding to each variation direction (Subtype) of each chromosome coordinate (Position), and perform fitting to the vaf of the variant population according to an inverse Gamma distribution.

4.3 Bioinformation analysis—Variation recognition:—First Trimmomatic (v0.36) software was used to remove adapters and low-quality sequencing products (reads). Then BWA aligner (v0.7.17) software was used to align the clean reads to the human hg19 reference genome. Next, Picard (v2.23.0) software was used to classify and remove duplications. VarDict (v1.5.1) software was used for identification and detection of SNV and InDel, and FreeBayes (v1.2.0) was used for complex mutations. The filtering of QC data such as mutation quality and chain preference was listed in the original variation list. In addition, variations in low-complex repeats and fragment repeats that match the low-mapping regions defined in ENCOD, as well as variations in the list of sequencing-specific errors (SSEs) developed and validated internally, were removed.

Screening for gene variants in tumor tissues:—First, variants were filtered from germline or hematopoietic sources. Variants that meet any of the following criteria were filtered out: (1) The variant frequency (VAF) from the peripheral blood is not less than 5%, or (2) the variant came from the peripheral blood, VAF value is less than 5%, but the VAF value does not exceed a 5 times relationship comparing to the VAF of the matched tissue sample at the point, or (3) the variant can be found in the public gnomAD population database, which has a small allele frequency (MAF) and is not less than 2%.

The remaining gene variants were further filtered by quality conditions. When screening tumor tissue variants, each variant was supported by at least 5 reads. The detection limit of SNV was 4%, and the detection limit of InDel was 5%. These are respectively used as the conditions for screening tumor tissue variants.

Screening for gene variants in plasma:—In this embodiment, the detection of the plasma variant signal only tracked the variant detected in the tumor tissue that met the above-mentioned detection criteria. The variant information (VSMj, TSMj) was obtained of variatnt j (Varient j), and the combined model of the variant was called according to the coordinates and direction of the variant. The combined model includes a population frequency Pzero at vaf=0 and the distribution (at vaf≠0). N times of samplings (N=10000) was performed by applying Monte Carlo Simulation sampling method, generate N×Pzero number of vaf=0, and generate N×(1−Pzero) number of random vaf based on the variant model part, respectively. Each of the N number of vaf were used as apriori noise frequency, to calculate the probability of the variant signal (VSMj, TSMj) coming from noise according to the binomial distribution. The probability was calculated by,

Pi=0, if vafᵢ=0

Pi=1−binomial(n≤VSMⱼ−1|TSMⱼ,vafᵢ) if vaf≠0

Then, the N number of calculation results were combined, and further calculated as a summed average of Pi. The summed average P is expressed as,

P=Σ₁^{N}Pi

The summed average P is a measure of the significance of the single point variation. The significance threshold of a single variation is defined as cutoff1=0.05. When the single variant value P≤0.05, the P value of the variation was included in the multi-variant combination analysis; otherwise, it was not included. The MRD sample judgment threshold was defined as cutoff2=0.01. That is, when the P value obtained by multi-variation joint confidence probability analysis was ≤0.01, it was considered that the degree of variation of the sample was significantly different from the noise, and it was judged as MRD+; when the P>0.01, the variant of the sample was considered to have no significant difference from the noise, and it was judged as MRD−.

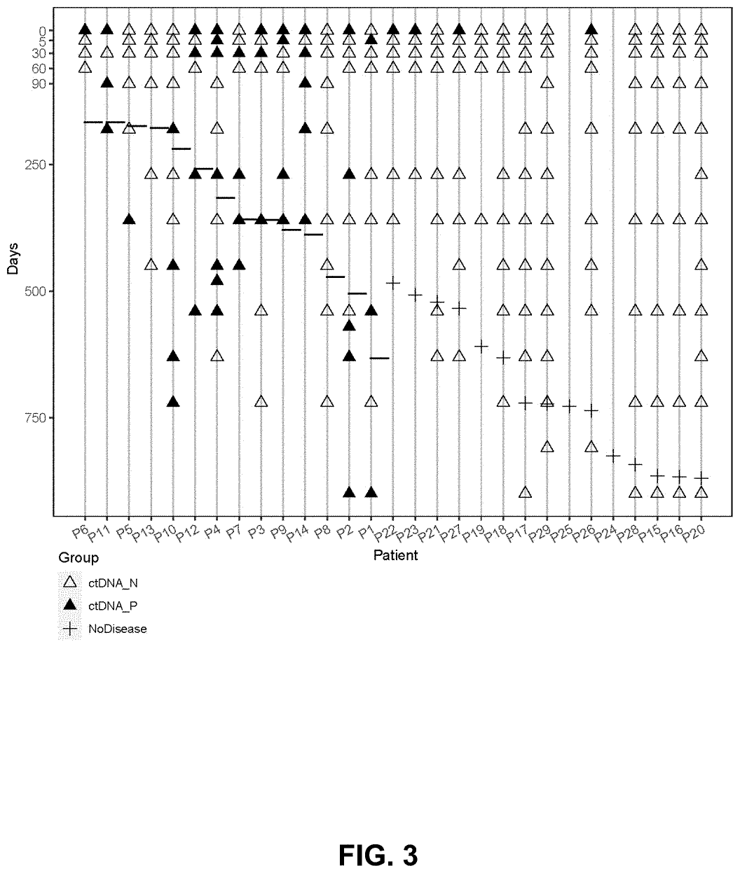

4.4 Analysis of results—Of the 27 patients (as shown in FIG. 3), 14 patients experienced relapse during follow-up. The median DFS of patients who relapsed was 337 days (166-632 days). 13 patients did not relapse during follow-up. The patient's relapse status and stage does not show a significant correlation (Table 3.1). In 13 patients who did not relapse, the ctDNA test results were negative during multiple follow-ups after surgery, and the specificity was 100% (CI95, 77.19%-100%). The proportion of 14 patients with relapse who tested positive one month after surgery was 35.7% (5/14). During the follow-up, 11 patients tested positive for ctDNA, with a sensitivity of 78.6% (CI95, 52.41%-92.43%). In 10 cases, the ctDNA signal was detected before the imaging examination progressed, and the median leadtime was 231 days (39-358 days). The results of this case show that the analysis algorithm based on the combined model Monte Carlo sampling had a high consistency between the detection of ctDNA and the relapse of the patient's tumor, and this technology platform well in predicting the relapse of the patient.

TABLE 4
Stages of 27 patients and their positive ctDNA
detection status during follow-up

| Patients | status | DFS | STAGE |
|---|---|---|---|
| P1 | relapse | 632.00 | StageI |
| P2 | relapse | 505.00 | StageIII |
| P3 | relapse | 359.00 | StageII |
| P4 | relapse | 315.00 | StageIII |
| P5 | relapse | 174.00 | StageI |
| P6 | relapse | 166.00 | StageII |
| P7 | relapse | 358.00 | StageII |
| P8 | relapse | 472.00 | StageI |
| P9 | relapse | 379.00 | StageIII |
| P10 | relapse | 219.00 | StageI |
| P11 | relapse | 166.00 | StageII |
| P12 | relapse | 258.00 | StageII |
| P13 | relapse | 177.00 | StageII |
| P14 | relapse | 388.00 | StageII |
| P15 | Not | 865.00 | StageI |
| | relapse | | |
| P16 | Not | 867.00 | StageI |
| | relapse | | |
| P17 | Not | 721.00 | StageII |
| | relapse | | |
| P18 | Not | 631.00 | StageII |
| | relapse | | |
| P19 | Not | 609.00 | StageII |
| | relapse | | |
| P20 | Not | 870.00 | StageIII |
| | relapse | | |
| P21 | Not | 522.00 | StageIII |
| | relapse | | |
| P22 | Not | 484.00 | StageII |
| | relapse | | |
| P23 | Not | 508.00 | StageIII |
| | relapse | | |
| P24 | Not | 736.00 | StageII |
| | relapse | | |
| P25 | Not | 534.00 | StageII |
| | relapse | | |
| P26 | Not | 843.00 | StageI |
| | relapse | | |
| P27 | Not | 722.00 | StageII |
| | relapse | | |

TABLE 5
PanelP1 gene list

| | | |
|---|---|---|
| AKT1 | FBXW7 | NRAS |
| ALK | FGFR1 | NTRK1 |
| APC | FGFR2 | PDGFRA |
| BRAF | FGFR3 | PIK3CA |
| CTNNB1 | KIT | PTEN |
| DDR2 | KRAS | RET |
| EGFR | MAP2K1 | ROS1 |
| ERBB2 | MET | SMAD4 |
| ERBB4 | NOTCH1 | STK11 |
| TP53 | UGT1A1 | |

TABLE 6
PanelP2 gene list

| | | | | | | | |
|---|---|---|---|---|---|---|---|
| ABCA13 | CACNA2D1 | DPP6 | GNAQ | MAP3K1 | PARD6B | RPF2 | TNIK |
| ABCA8 | CALD1 | DPYD | GNAS | MAP3K13 | PARK2 | RPRD1A | TNKS |
| ABCB1 | CALM2 | DSCAM | GPAT3 | MAP3K4 | PARP1 | RPS6KB1 | TNRC18 |
| ABCC2 | CALR | E2F3 | GPC4 | MAP4K3 | PARP2 | RPTOR | TOP1 |
| ABCC9 | CARD11 | EBP | GPM6A | MAP4K5 | PARP3 | RRM1 | TOP2B |
| ABL1 | CASP8 | EED | GRB10 | MAPK1 | PARP8 | RRP1B | TP53 |
| ACADSB | CAST | EGFR | GREM1 | MAPKAP1 | PAX3 | RUNX1 | TP63 |
| ACOT13 | CBFB | EIF1AX | GRIK2 | MAPKBP1 | PAX5 | RWDD1 | TPH1 |
| ACRC | CBL | EIF4E | GRIN2A | MARK1 | PBRM1 | RYBP | TPM1 |
| ADCY8 | CBR3 | EIF4G3 | GSK3B | MARK3 | PDCD1 | RYR2 | TRA2A |
| ADGRG6 | CBR4 | ELFN1 | GSKIP | MAX | PDCD1LG2 | SASH1 | TRAF7 |
| AGAP1 | CCDC157 | ELMOD2 | GSTA1 | MCL1 | PDE4D | SCOC | TRIM24 |
| AK7 | CCDC18 | EML4 | GSTM1 | MDC1 | PDGFRA | SDHA | TRIM25 |
| AKT1 | CCND1 | ENOSF1 | GSTP1 | MDM2 | PDGFRB | SDHAF2 | TSC1 |
| AKT2 | CCND2 | ENSA | GUCY1A2 | MDM4 | PDPK1 | SDHB | TSC2 |
| AKT3 | CCND3 | EP300 | H3F3A | MED12 | PDS5A | SDHC | TSHR |
| ALDH5A1 | CCNE1 | EPCAM | HAUS2 | MED12L | PFKP | SDHD | TSN |
| ALG9 | CD274 | EPG5 | HAUS6 | MED14 | PGBD1 | SEL1L3 | TTC1 |
| ALK | CD40 | EPHA3 | HCAR2 | MED19 | PGR | SEMA3C | TTC6 |
| ALOX12B | CD74 | EPHA5 | HDGFRP3 | MEF2BNB- | PGRMC2 | SEMA3E | TTN |
| | | | | MEF2B | | | |
| ALS2CR11 | CD79A | EPHA7 | HERC6 | MEIS1 | PHF20 | SERTAD4 | TUBD1 |
| AMBRA1 | CD79B | EPHB1 | HEY1 | MEN1 | PIGF | SETD2 | TXNDC16 |
| AMER1 | CDA | EPYC | HGF | MET | PIK3C2G | SF3B1 | TXNRD1 |
| ANAPC7 | CDC73 | ERBB2 | HIST1H1C | METTL9 | PIK3C3 | SFXN4 | U2AF1 |
| ANKRD28 | CDCA8 | ERBB3 | HIST1H3B | MITF | PIK3CA | SH2D1A | UBAP2L |
| ANKRD46 | CDH1 | ERBB4 | HLA-A | MLH1 | PIK3CB | SHQ1 | UBE2E3 |
| ANO1 | CDK12 | ERCC1 | HLA-B | MLH3 | PIK3CD | SHROOM3 | UBE4A |
| APAF1 | CDK4 | ERCC2 | HLA-C | MMP16 | PIK3CG | SIMC1 | UBN2 |
| APC | CDK6 | ERCC3 | HMCN1 | MMP3 | PIK3R1 | SIPA1L2 | UBXN7 |
| APOL2 | CDK8 | ERCC4 | HNF1A | MPL | PIK3R2 | SKA3 | UGT1A1 |
| APOPT1 | CDKL3 | ERG | HNF4A | MRE11A | PIK3R3 | SLC13A1 | ULK2 |
| AQR | CDKN1A | ERI1 | HOMER1 | MRPL19 | PIM1 | SLC22A2 | ULK4 |
| AR | CDKN1B | ERRFI1 | HRAS | MS4A13 | PKHD1 | SLC25A13 | UMPS |
| ARAF | CDKN2A | ESR1 | HSD17B11 | MSANTD3- | PLCG2 | SLC30A5 | UPF2 |
| | | | | TMEFF1 | | | |
| ARHGAP26 | CDKN2B | ETV1 | HSD3B1 | MSH2 | PLEKHA1 | SLC31A1 | USP11 |
| ARHGAP4 | CDKN2C | ETV4 | HSPA1B | MSH3 | PLEKHH2 | SLC35B1 | USP34 |
| ARHGAP6 | CDO1 | ETV5 | HSPA4 | MSH6 | PLXNC1 | SLC7A8 | USP9Y |
| ARHGEF12 | CEBPA | ETV6 | HSPA5 | MTF1 | PMS1 | SLC9C2 | UTS2 |
| ARHGEF3 | CEP120 | EWSR1 | HSPH1 | MTF2 | PMS2 | SLCO1B1 | UTY |
| ARID1A | CEP290 | EXOSC8 | HTT | MTHFR | PNO1 | SLCO1B3 | VEGFA |
| ARID1B | CFAP221 | EZH2 | HYOU1 | MTOR | POLA1 | SLIT1 | VHL |
| ARID2 | CFAP53 | EZR | IARS | MTR | POLD1 | SLX4 | VSIG10 |
| ARID4A | CHD1 | FAM149A | ICOSLG | MTRR | POLE | SMAD2 | WDR5 |
| ARID5B | CHD2 | FAM153B | ID2 | MUTYH | POSTN | SMAD3 | WHSC1 |
| ARL13B | CHEK1 | FAM161A | ID3 | MYADM | PPARG | SMAD4 | WHSC1L1 |
| ARL4A | CHEK2 | FAM175A | IDH1 | MYB | PPP1R21 | SMARCA4 | WT1 |
| ARL6IP6 | CHRM3 | FAM184B | IDH2 | MYC | PPP2R1A | SMARCB1 | XIAP |
| ARMC5 | CHURC1- | FAM20A | IGF1 | MYCL | PRDM1 | SMO | XPC |
| | FNTB | | | | | | |
| ASB11 | CIC | FAM46C | IGF1R | MYCN | PRELID3B | SNX6 | XPO1 |
| ASH1L | CLASP2 | FANCA | IGF2 | MYD88 | PREX2 | SOCS1 | XRCC1 |
| ASPH | CLEC16A | FANCC | IKBKE | MYO10 | PRKAR1A | SOD2 | XRCC2 |
| ASXL1 | CLEC9A | FANCD2 | IKZF1 | MYOD1 | PRKCI | SOX17 | YAP1 |
| ASXL2 | CNKSR3 | FANCF | IL10 | MYOM1 | PRKDC | SOX2 | YLPM1 |
| ATG3 | CNOT8 | FANCG | IL13RA1 | MZT2A | PRPF39 | SOX9 | YWHAE |
| ATG4C | COL15A1 | FAS | IL7R | NAB1 | PRPF4 | SPEN | ZBBX |
| ATIC | COX18 | FAT1 | IMPG1 | NAMPT | PTCH1 | SPOP | ZBTB40 |
| ATM | CPS1 | FBXO11 | INHBA | NAPG | PTEN | SRC | ZDHHC17 |
| ATP6V0A1 | CREBBP | FBXW7 | INPP4A | NAV1 | PTK2 | SRSF3 | ZDHHC20 |
| ATP6V0A2 | CRKL | FGF10 | INPP4B | NBAS | PTPN11 | SRY | ZMYM2 |
| ATP6V0A4 | CRLF2 | FGF16 | IRF4 | NBEAL1 | PTPN4 | STAB2 | ZMYM4 |
| ATP6V0E1 | CSF1R | FGF19 | IRF6 | NBN | PTPRD | STAG2 | ZNF195 |
| ATP8A1 | CSF3R | FGF3 | IRF8 | NCOA6 | PTPRJ | STARD4 | ZNF2 |
| ATR | CTAGE5 | FGF4 | IRS2 | NCOR1 | PTPRS | STAT3 | ZNF280D |
| ATRX | CTCF | FGF6 | ITGAL | NEDD4L | PTPRT | STK11 | ZNF283 |
| AURKA | CTLA4 | FGFR1 | JAK1 | NEO1 | PURA | STMN1 | ZNF367 |
| AURKB | CTNNB1 | FGFR2 | JAK2 | NF1 | RAB2B | STRBP | ZNF711 |
| AXIN1 | CTSC | FGFR3 | JAK3 | NF2 | RABGAP1L | STT3A | ZNF805 |
| AXIN2 | CUL3 | FGFR4 | JUN | NFE2L2 | RAC1 | STYX | ZNF91 |
| AXL | CXCL8 | FH | KDM5A | NFKBIA | RAD21 | SUCLG1 | ZZZ3 |
| B2M | CXCR4 | FLCN | KDM5C | NFXL1 | RAD50 | SUFU | |
| BAP1 | CYBA | FLI1 | KDM6A | NKAP | RAD51 | SUGCT | |
| BARD1 | CYFIP1 | FLOT1 | KDR | NKX2-1 | RAD51B | SUZ12 | |
| BCAS1 | CYLD | FLT1 | KEAP1 | NLRP7 | RAD51C | SYK | |
| BCL2 | CYP19A1 | FLT3 | KIAA1210 | NOTCH1 | RAD51D | SYNE2 | |
| BCL2L1 | CYP2B6 | FLT4 | KIAA1841 | NOTCH2 | RAD52 | TAF15 | |
| BCL2L11 | CYP2C19 | FMNL2 | KIT | NOTCH3 | RAD54L | TAOK3 | |
| BCL6 | CYP2C8 | FMO1 | KLF4 | NOTCH4 | RAF1 | TARBP1 | |
| BCOR | CYP2D6 | FMR1 | KMT2A | NPM1 | RALGAPB | TBC1D8B | |
| BCR | DARS2 | FNBP4 | KMT2C | NR1I3 | RAP2B | TBCD | |
| BIRC3 | DAXX | FOLH1B | KMT2D | NRAS | RARA | TBX3 | |
| BIVM- | DCHS2 | FOXAl | KPNA4 | NRG1 | RASA1 | TECPR2 | |
| ERCC5 | | | | | | | |
| BLM | DDR1 | FOXL2 | KPNB1 | NRG4 | RB1 | TENM3 | |
| BMPR1A | DDR2 | FOXO1 | KRAS | NSD1 | RBM10 | TERT | |
| BRAF | DDX19B | FOXP1 | KTN1 | NT5C2 | RBM27 | TERT- | |
| | | | | | | promoter | |
| BRCA1 | DDX58 | FPGT- | LAMA3 | NTHL1 | RECQL4 | TET1 | |
| | | TNNI3K | | | | | |
| BRCA2 | DEPDC5 | FUBP1 | LATS1 | NTRK1 | REL | TET2 | |
| BRD4 | DHFR | FUS | LATS2 | NTRK2 | RET | TFDP1 | |
| BRIP1 | DIAPH1 | FXR1 | LEPR | NTRK3 | RFC1 | TFRC | |
| BRMS1L | DIAPH2 | GABRP | LMO1 | NUDT13 | RFWD2 | TGFBR1 | |
| BRS3 | DICER1 | GALNT12 | LNPEP | NUP85 | RHOA | TGFBR2 | |
| BTF3 | DIS3 | GALNT14 | LONRF3 | NUP93 | RHOT1 | TMEM126B | |
| BTG1 | DLC1 | GANC | LRP2 | OSBP | RIC1 | TMEM127 | |
| BTK | DMXL1 | GATA1 | LRRC16A | OTOGL | RICTOR | TMEM132D | |
| C22orf23 | DNAJB1 | GATA2 | LRRC34 | OTOS | RIPK2 | TMEM67 | |
| C5orf15 | DNAJC11 | GATA3 | LYN | P2RY8 | RIT1 | TMPRSS15 | |
| C5orf42 | DNMT1 | GIPC1 | MALRD1 | PAK1 | RNF112 | TMPRSS2 | |
| C7orf66 | DNMT3A | GLI1 | MALT1 | PAK7 | RNF19A | TMTC4 | |
| C8orf34 | DNMT3B | GMEB1 | MAP2K1 | PALB2 | RNF43 | TNFAIP3 | |
| CAB39 | DOCK11 | GNA11 | MAP2K2 | PAPOLG | ROBO1 | TNFRSF14 | |
| CACNA1E | DOT1L | GNA13 | MAP2K4 | PAQR8 | ROS1 | TNFSF13B | |

TABLE 7
PanelP3 gene list

| | | | | | |
|---|---|---|---|---|---|
| ABALON | CHEK2 | GLI3 | MEN1 | PTPN23 | TP53 |
| ABCA1 | CHST3 | GLO1 | MEP1B | PTPRB | TP63 |
| ABCA13 | CIC | GLRX | MET | PTPRD | TP73 |
| ABCA8 | CIITA | GLRX2 | METAP1 | PTPRG | TPBG |
| ABCB1 | CLEC1B | GMEB1 | MFSD11 | PTPRJ | TPH1 |
| ABCB11 | CLEC4G | GNA11 | MGA | PTPRK | TPH2 |
| ABCC1 | CLIC1 | GNA13 | MGAM | PTPRT | TPI1 |
| ABCC11 | CLIP1 | GNAQ | MGMT | PTTG1 | TPM3 |
| ABCC2 | CLK3 | GNAS | MIF | PURA | TPM4 |
| ABCC3 | CLTC | GOLGA5 | MIF-AS1 | PUS1 | TPMT |
| ABCC4 | CMPK1 | GOPC | MIR1206 | PYGM | TPP1 |
| ABCC5 | CNKSR3 | GPC1 | MIR1273H | PYROXD1 | TRA2A |
| ABCC6 | CNOT1 | GPC3 | MIR1307 | QKI | TRAF2 |
| ABCC9 | CNOT8 | GPI | MIR146A | RAB27A | TRAF7 |
| ABCG2 | COL11A1 | GPM6A | MIR2053 | RABGAP1L | TRIM24 |
| ABL1 | COL18A1 | GPX5 | MIR27A | RAC1 | TRIM27 |
| ABL2 | COL1A1 | GPX6 | MIR300 | RAD21 | TRIM33 |
| ACADL | COL1A2 | GPX7 | MIR3184 | RAD50 | TRMT61B |
| ACADSB | COL4A1 | GRB7 | MIR323B | RAD51 | TRPS1 |
| ACE | COL4A5 | GREM1 | MIR423 | RAD51B | TRPV4 |
| ACO1 | COL6A2 | GRIK1 | MIR449B | RAD51C | TRRAP |
| ACO2 | COX18 | GRIN2A | MIR492 | RAD51D | TSC1 |
| ACOT13 | CPA1 | GRM3 | MIR577 | RAD51L3-RFFL | TSC2 |
| ACP5 | CPA2 | GRM8 | MIR604 | RAD52 | TSG101 |
| ACPP | CPA4 | GSG2 | MIR618 | RAD54L | TSHR |
| ACSM2A | CPB2 | GSK3B | MIR6752 | RAF1 | TSN |
| ACSS2 | CRABP2 | GSN | MIR6759 | RALA | TSPAN31 |
| ACTG1 | CRBN | GSR | MITD1 | RALB | TSPYL2 |
| ACTR8 | CREB1 | GSS | MITF | RAMP3 | TTC36 |
| ACVR1 | CREBBP | GSTA1 | MKI67 | RAN | TTF1 |
| ACVR1B | CRHBP | GSTA3 | MKRN1 | RANBP2 | TTK |
| ACVR2A | CRKL | GSTM1 | MLH1 | RARA | TTLL2 |
| ACVR2B | CRLF2 | GSTO1 | MLH3 | RARB | TTLL5 |
| ADAM22 | CRTC1 | GSTP1 | MLL2 | RARG | TTR |
| ADAM29 | CRYZ | GSTT1 | MLL3 | RASAL1 | TUBB1 |
| ADAMTS6 | CS | GUSB | MLLT1 | RASGRF1 | TUBB3 |
| ADAMTSL1 | CSDE1 | GXYLT1 | MLLT10 | RASGRF2 | TUBD1 |
| ADAMTSL4 | CSF1R | H19 | MLLT3 | RASSF1 | TXNRD1 |
| ADCY10 | CSF2RB | H3F3A | MLLT4 | RASSF1-AS1 | TYMP |
| ADGRA2 | CSF3R | H3F3AP4 | MMAB | RB1 | TYMS |
| ADH1B | CSMD3 | H3F3B | MMP11 | RBM10 | TYRO3 |
| ADH1C | CSNK1A1 | HADH | MMP13 | RBM27 | U2AF1 |
| ADHFE1 | CSNK2A1 | HAGH | MMP16 | RBP2 | UBA1 |
| ADIPOQ | CST6 | HAL | MMP8 | RBP4 | UBC |
| ADIPOQ-AS1 | CTAGE5 | HAS3 | MMP9 | RECQL | UBE2D1 |
| ADORA2A-AS1 | CTCF | HAT1 | MONO-27 | RECQL4 | UBE2D2 |
| ADRB1 | CTNNA1 | HAUS2 | MOV10L1 | REL | UBE2E3 |
| ADRB2 | CTNNB1 | HCAR2 | MPL | RELA | UBE2I |
| ADRB3 | CTNND1 | HCN4 | MRE11A | RET | UBE3C |
| ADSS | CTSA | HDAC1 | MRPL13 | REV3L | UBR3 |
| AFF1 | CTSD | HDAC2 | MRPL19 | RGS5 | UBR5 |
| AFF4 | CTSE | HDAC8 | MSH2 | RHBDF2 | UGT1A1 |
| AGO1 | CTSS | HERPUD1 | MSH3 | RHEB | UGT1A10 |
| AGPAT9 | CUL3 | HEXB | MSH5 | RHOA | UGT1A3 |
| AGTRAP | CUX1 | HEY1 | MSH5-SAPCD1 | RHOBTB2 | UGT1A4 |
| AHR | CXCL1 | HGF | MSH6 | RHOC | UGT1A5 |
| AIP | CXCL3 | HIC1 | MSI2 | RHOT1 | UGT1A6 |
| AK7 | CXCL8 | HIF1A | MSN | RICTOR | UGT1A7 |
| AKAP9 | CXCR4 | HIP1 | MST1R | RIPK2 | UGT1A8 |
| AKNA | CXXC4 | HIST1H1C | MTAP | RNASE2 | UGT1A9 |
| AKR1B1 | CYB561D2 | HIST1H2BD | MTBP | RNF128 | ULBP3 |
| AKR1C2 | CYBA | HIST1H3A | MTF1 | RNF146 | ULK3 |
| AKR1C3 | CYFIP1 | HIST1H3B | MTHFD1 | RNF19A | ULK4 |
| AKR1C4 | CYLD | HIST1H3C | MTHFR | RNF43 | UMPS |
| AKT1 | CYP19A1 | HIST1H3D | MTOR | ROCK1 | UPF2 |
| AKT2 | CYP1A1 | HIST1H3E | MTR | RORC | UPP1 |
| AKT3 | CYP1A2 | HIST1H3F | MTRR | ROS1 | USMG5 |
| AKTIP | CYP1B1 | HIST1H3G | MUTYH | RPA4 | USP25 |
| ALB | CYP2A13 | HIST1H3H | MYADM | RPS6KA3 | USP6 |
| ALDH2 | CYP2A6 | HIST1H3I | MYB | RPS6KB1 | USP9X |
| ALDOA | CYP2A7 | HIST1H3J | MYBL2 | RPS6KC1 | UTY |
| ALDOB | CYP2B6 | HIST1H4A | MYC | RPTOR | VEGFA |
| ALDOC | CYP2C19 | HK1 | MYCL | RRAGC | VEGFC |
| ALG9 | CYP2C8 | HK2 | MYCN | RRAS2 | VEGFD |
| ALK | CYP2C9 | HK3 | MYD88 | RRM1 | VHL |
| ALOX12 | CYP2D6 | HLA-A | MYH9 | RRM2 | VRK2 |
| ALOX12B | CYP2D7 | HLA-B | MYO10 | RRP1B | VSIG10 |
| ALS2CL | CYP2E1 | HLA-C | MYOD1 | RSPO1 | VWF |
| ALS2CR11 | CYP2R1 | HLA-DOA | NAB1 | RTEL1 | WARS |
| AMER1 | CYP3A4 | HLA-DOB | NAB2 | RUNX1 | WAS |
| AMPD1 | CYP3A5 | HLA-DPA1 | NACC1 | RUNX1T1 | WEE1 |
| AMPH | CYP46A1 | HLA-DQA1 | NAGA | RUNX3 | WHSC1 |
| ANK1 | CYP4B1 | HLA-DQB1 | NALCN | RUSC1 | WHSC1L1 |
| ANKRA2 | D2HGDH | HLA-DRA | NAMPT | RXRA | WISP3 |
| ANKRD46 | DAB2IP | HLA-DRB1 | NAT2 | RYR2 | WNT1 |
| ANO1 | DAXX | HLA-G | NAV3 | S100A4 | WNT11 |
| ANTXR2 | DAZL | HMGCR | NBN | SAMD9L | WNT4 |
| AOX1 | DBT | HMGXB3 | NCAM2 | SASH1 | WRAP53 |
| AP4B1-AS1 | DCK | HN1 | NCOA1 | SBDS | WRN |
| APAF1 | DCTN1 | HNF1A | NCOA4 | SCD | WT1 |
| APC | DDIT3 | HNF1B | NCOA6 | SCN10A | WWC3 |
| APCS | DDR1 | HNF4A | NCOR1 | SCUBE2 | WWP1 |
| APEX1 | DDR2 | HNRNPA2B1 | NCOR2 | SDC4 | WWTR1 |
| APOB | DDX27 | HNRNPH1 | NDUFS1 | SDCBP | XBP1 |
| APOE | DDX3X | HOOK3 | NEDD4 | SDHA | XDH |
| APOPT1 | DDX6 | HOTAIR | NEDD4L | SDHAF2 | XIRP1 |
| AQP9 | DEAR | HOXA13 | NEK8 | SDHB | XPA |
| AR | DENND1A | HOXB13 | NEO1 | SDHC | XPC |
| ARAF | DEPDC5 | HOXB4 | NEU2 | SDHD | XPO1 |
| AREG | DERL3 | HOXC4 | NF1 | SEL1L3 | XPO5 |
| ARFRP1 | DHFR | HPDL | NF2 | SELL | XRCC1 |
| ARHGAP19 | DIAPH1 | HPGDS | NFASC | SEMA3B | XRCC3 |
| ARHGAP19- | DICER1 | HRAS | NFATC2 | SEMA3C | XRCC5 |
| SLIT1 | | | | | |
| ARHGAP4 | DIDO1 | HSD17B4 | NFE2L2 | SEMA3F | XRCC6 |
| ARHGAP6 | DIS3 | HSD3B1 | NFKBIA | SENP3-EIF4A1 | YAP1 |
| ARHGAP9 | DLAT | HSP90AA1 | NFXL1 | SENP5 | ZADH2 |
| ARHGEF7 | DLD | HSPA1B | NKX2-1 | SERP2 | ZBBX |
| ARHGEF7-AS2 | DLG4 | HSPA4 | NLGN4X | SERPINA7 | ZBTB17 |
| ARID1A | DLG5 | HSPA5 | NLRP3 | SERPINB3 | ZBTB2 |
| ARID1B | DLL3 | HSPA8 | NME1 | SETBP1 | ZC3H13 |
| ARID2 | DLST | HYOU1 | NME1-NME2 | SETD1B | ZDHHC17 |
| ARID4A | DMD | IARS | NME2 | SETD2 | ZFHX3 |
| ARID5B | DNAJB1 | ID2 | NMRAL1 | SETD3 | ZFHX4 |
| ARL6IP6 | DNMT1 | ID3 | NNT | SETD6 | ZIC3 |
| ARMC5 | DNMT3A | IDH1 | NOS3 | SETD8 | ZIM2 |
| ARMS2 | DOCK11 | IDH2 | NOTCH1 | SF3B1 | ZMIZ1 |
| ARNT | DOCK2 | IDH3A | NOTCH2 | SFN | ZMYND10 |
| ARPC2 | DOT1L | IDH3B | NOTCH3 | SFRP1 | ZNF189 |
| ARRDC3 | DPEP1 | IDH3G | NOTCH4 | SFRP2 | ZNF2 |
| ASH1L | DPYD | IFNL3 | NPC1 | SGK1 | ZNF217 |
| ASPM | DROSHA | IGF1 | NPFF | SH2B3 | ZNF226 |
| ASXL1 | DSCAM | IGF1R | NPM1 | SH2D1A | ZNF276 |
| ASXL2 | DSE | IGF2 | NPY | SH3GL2 | ZNF331 |
| ATAD3B | DST | IGSF10 | NQO1 | SHISA5 | ZNF444 |
| ATAD5 | DTYMK | IGSF3 | NQO2 | SHMT1 | ZNF521 |
| ATF1 | DUSP2 | IKBKB | NR1I2 | SHOX | ZNF703 |
| ATIC | DVL1 | IKBKE | NR1I3 | SHROOM3 | ZNF711 |
| ATM | DYNC2H1 | IKZF1 | NR-21 | SIGLEC7 | ZNF805 |
| ATP10B | E2F1 | IKZF3 | NR-24 | SIPA1L2 | ZNRF3 |
| ATP5S | ECT2L | IL13 | NR3C1 | SIRPA | ZRSR2 |
| ATP7A | EED | IL16 | NR3C2 | SIRT2 | ZZZ3 |
| ATP7B | EGF | IL17F | NR4A3 | SLC10A1 | |
| ATP9B | EGFR | IL1B | NRAS | SLC10A2 | |
| ATR | EGFR-AS1 | IL1RL1 | NRG1 | SLC16A1 | |
| ATRX | EGR1 | IL2 | NSD1 | SLC16A3 | |
| AURKA | EIF1AX | IL20RA | NT5C1A | SLC16A7 | |
| AURKB | EIF3A | IL21R | NT5C2 | SLC16A8 | |
| AXIN1 | EIF4A1 | IL21R-AS1 | NT5C3A | SLC19A1 | |
| AXIN2 | EIF4A2 | IL23R | NTRK1 | SLC22A1 | |
| AXL | EIF4EBP1 | IL6ST | NTRK2 | SLC22A12 | |
| AZGP1 | EIF4G3 | IL7R | NTRK3 | SLC22A16 | |
| AZU1 | ELMO1 | ING1 | NUDC | SLC22A2 | |
| B2M | ELMO1-AS1 | ING2 | NUDT15 | SLC22A4 | |
| B9D2 | EML4 | ING3 | NUDT2 | SLC28A1 | |
| BAG1 | ENO1 | ING5 | NUP85 | SLC28A2 | |
| BAI3 | ENO2 | INHBA | NUP93 | SLC28A3 | |
| BAIAP2L1 | ENO3 | INPP4B | NUTM1 | SLC31A1 | |
| BAK1 | ENOSF1 | INPP5D | OBSCN | SLC34A2 | |
| BAP1 | EP300 | INS-IGF2 | OGDH | SLC45A3 | |
| BARD1 | EP400 | IPO7 | OTOP1 | SLC5A8 | |
| BARX1 | EPAS1 | IQGAP1 | OTOS | SLC6A4 | |
| BAT-25 | EPCAM | IRAK1 | P2RY8 | SLC7A8 | |
| BAT-26 | EPHA2 | IRF1 | PAH | SLC9A9 | |
| BAX | EPHA3 | IRF2 | PAK1 | SLCO1B1 | |
| BAZ2B | EPHA4 | IRF4 | PAK2 | SLCO1B3 | |
| BCAT1 | EPHA5 | IRF6 | PAK3 | SLIT1 | |
| BCL10 | EPHA7 | IRF8 | PALB2 | SLIT2 | |
| BCL11B | EPHB1 | IRS1 | PALLD | SLX4 | |
| BCL2 | EPHB4 | IRS2 | PAPOLG | SMAD2 | |
| BCL2L1 | EPHB6 | ITCH | PAQR8 | SMAD3 | |
| BCL2L11 | EPHX1 | ITGA2B | PARK2 | SMAD4 | |
| BCL2L2 | EPHX2 | ITGA4 | PARP1 | SMAD7 | |
| BCL2L2-PABPN1 | EPRS | ITGA5 | PARP2 | SMARCA1 | |
| BCL6 | EPS15 | ITGAL | PAX5 | SMARCA4 | |
| BCOR | ERAP2 | ITGAV | PBRM1 | SMARCB1 | |
| BCORL1 | ERBB2 | ITGAX | PC | SMARCD1 | |
| BCR | ERBB3 | ITGB2 | PCK1 | SMN1 | |
| BCYRN1 | ERBB4 | ITPA | PCLO | SMN2 | |
| BID | ERC1 | JAG1 | PCM1 | SMO | |
| BIRC3 | ERCC1 | JAK1 | PCMTD1 | SMS | |
| BIRC5 | ERCC2 | JAK2 | PCNA | SMYD2 | |
| BIVM-ERCC5 | ERCC3 | JAK3 | PDCD1 | SNAPC5 | |
| BLM | ERCC4 | JMJD6 | PDCD1LG2 | SNCAIP | |
| BLNK | ERCC5 | JUN | PDE10A | SNRNP200 | |
| BMPR1A | ERCC6 | KARS | PDE11A | SNX6 | |
| BMX | ERCC6- | KAT6A | PDE4B | SOCS1 | |
| | PGBD3 | | | | |
| BRAF | EREG | KAT6B | PDE4DIP | SOD2 | |
| BRCA1 | ERG | KCNB2 | PDE5A | SOS2 | |
| BRCA2 | ERI1 | KCNJ2 | PDE6C | SOX1 | |
| BRD4 | ERP44 | KDM4D | PDGFA | SOX17 | |
| BRD7 | ERRFI1 | KDM5A | PDGFB | SOX2 | |
| BRD9 | ESR1 | KDM5C | PDGFRA | SOX9 | |
| BRINP1 | ESR2 | KDM6A | PDGFRB | SPAG17 | |
| BRINP3 | ESRP1 | KDR | PDHA1 | SPC24 | |
| BRIP1 | ETF1 | KEAP1 | PDHB | SPEN | |
| BRS3 | ETS1 | KEL | PDHX | SPG7 | |
| BRWD1 | ETV1 | KHDRBS2 | PDIA2 | SPOP | |
| BSG | ETV4 | KIAA1210 | PDK1 | SPRY2 | |
| BTF3 | ETV5 | KIAA1432 | PDK2 | SPRY4 | |
| BTG1 | ETV6 | KIF15 | PDK3 | SPTA1 | |
| BTG2 | EWSR1 | KIF5B | PDK4 | SRC | |
| BTK | EXO1 | KIR3DX1 | PDP1 | SRCAP | |
| BTN3A1 | EXOSC8 | KIT | PDP2 | SRGAP3 | |
| BTRC | EXT1 | KITLG | PDPK1 | SRSF2 | |
| BUB1 | EXT2 | KLC1 | PDPN | SRXN1 | |
| BUB1B | EZH2 | KLF4 | PDPR | SS18 | |
| C11orf30 | EZR | KLF6 | PDXK | ST14 | |
| C1orf167 | F13A1 | KLHL12 | PEG3 | STAG2 | |
| C20orf96 | FAM131B | KLHL6 | PFKFB1 | STAT1 | |
| C22orf23 | FAM135B | KLLN | PFKFB2 | STAT2 | |
| C5orf42 | FAM149A | KMO | PFKFB3 | STAT3 | |
| C8orf34 | FAM153B | KMT2A | PFKFB4 | STAT4 | |
| C9orf72 | FAM46C | KMT2B | PFKL | STAT5A | |
| CA1 | FANCA | KMT2C | PFKM | STAT5B | |
| CA13 | FANCC | KMT2D | PFKP | STAT6 | |
| CA14 | FANCD2 | KPNA4 | PGAM1 | STIM1 | |
| CA2 | FANCE | KPNB1 | PGAP3 | STK11 | |
| CA4 | FANCF | KRAS | PGBD3 | STMN1 | |
| CA9 | FANCG | KRT14 | PGK1 | STOML1 | |
| CAB39 | FANCI | KRT18 | PGK2 | STRADA | |
| CACNA2D2 | FANCL | KRT19 | PGR | STRBP | |
| CACNA2D4 | FAP | KRT19P2 | PHF6 | STRN | |
| CADM1 | FAS | KRT8 | PHF8 | STS | |
| CALD1 | FASLG | KSR2 | PHKA2 | STT3A | |
| CALM2 | FASN | KTN1 | PHKA2-AS1 | STX5 | |
| CALM3 | FAT1 | L2HGDH | PHKG2 | SUCLA2 | |
| CALR | FAT2 | LAMA3 | PHOX2B | SUCLG1 | |
| CAMK1 | FAT3 | LAMP3 | PI4KA | SUCLG2 | |
| CAMK2A | FAT4 | LANCL1 | PIK3C2B | SUFU | |
| CAMK2N1 | FBXO11 | LARS2 | PIK3C2G | SUGCT | |
| CANT1 | FBXW7 | LATS1 | PIK3C3 | SULT1C4 | |
| CAPG | FCGR2A | LDHA | PIK3CA | SULT2B1 | |
| CARD11 | FCGR3A | LDHAL6A | PIK3CB | SUMO1 | |
| CARS | FCHSD1 | LDHAL6B | PIK3CG | SUV39H2 | |
| CASP2 | FCN1 | LDHB | PIK3R1 | SUZ12 | |
| CASP3 | FCN2 | LDHC | PIK3R2 | SYK | |
| CASP7 | FCRL1 | LEPR | PIM1 | SYN1 | |
| CASP8 | FDPS | LGALS3 | PINLYP | SYNE1 | |
| CASP9 | FECH | LGALS3BP | PKD1 | SYNE2 | |
| CAST, ERAP1 | FES | LGR5 | PKD2 | SYNPO2 | |
| CAV1 | FEV | LHCGR | PKHD1 | TAB1 | |
| CBFB | FGF10 | LIFR | PKLR | TACC1 | |
| CBL | FGF14 | LIG3 | PKM | TACC3 | |
| CBLB | FGF16 | LIG4 | PLA2G7 | TAF1 | |
| CBR1 | FGF19 | LIMD1 | PLAG1 | TAF15 | |
| CBR3 | FGF23 | LIPF | PLAT | TAF9 | |
| CBR4 | FGF3 | LMO1 | PLAU | TAGAP | |
| CBX5 | FGF4 | LOC100131626 | PLAUR | TARBP2 | |
| CBX7 | FGF6 | LOC100506321 | PLCB3 | TBC1D20 | |
| CCAT2 | FGFR1 | LOC100507346 | PLCG2 | TBC1D8B | |
| CCBL1 | FGFR2 | LOC101928414 | PLEKHA1 | TBL1XR1 | |
| CCDC178 | FGFR3 | LOC101929089 | PLEKHH2 | TBX3 | |
| CCL1 | FGFR4 | LOC101929829 | PLK1 | TBX5 | |
| CCNA1 | FH | LONRF3 | PLXNC1 | TCF3 | |
| CCNA2 | FHIT | LRIG3 | PMEL | TCF4 | |
| CCNB1 | FIBCD1 | LRP1B | PML | TCF7L1 | |
| CCNB2 | FKBP4 | LRP2 | PMM2 | TCF7L2 | |
| CCNB3 | FLCN | LRP5 | PMS1 | TCL1A | |
| CCND1 | FLI1 | LRP6 | PMS2 | TCN1 | |
| CCND2 | FLOT1 | LRRC34 | PNMT | TECPR2 | |
| CCND3 | FLT1 | LRRC4C | PNO1 | TEK | |
| CCNE1 | FLT3 | LSM14A | PNP | TEKT4 | |
| CCNE2 | FLT4 | LTA4H | PNRC1 | TEP1 | |
| CCR4 | FMO1 | LTF | POFUT2 | TERT | |
| CD180 | FMO3 | LY86 | POLB | TES | |
| CD1D | FN1 | LY96 | POLD1 | TET1 | |
| CD274 | FNTA | LYN | POLE | TET2 | |
| CD28 | FOLH1 | LZTR1 | POLH | TEX14 | |
| CD3EAP | FOLR2 | MACC1 | POLK | TFF1 | |
| CD40 | FOLR3 | MAD1L1 | POLR3H | TFG | |
| CD40LG | FOXA1 | MAGI1 | PON1 | TGFB1 | |
| CD44 | FOXL2 | MAGI2 | POT1 | TGFBR1 | |
| CD47 | FOXM1 | MAGI3 | POU5F1 | TGFBR2 | |
| CD55 | FOXO1 | MAGOHB | PPARD | TGFBR3 | |
| CD68 | FOXO3 | MALAT1 | PPARG | TGM2 | |
| CD74 | FOXP1 | MALT1 | PPFIBP1 | THADA | |
| CD79A | FPGS | MAOB | PPHLN1 | THRA | |
| CD79B | FRAS1 | MAP1B | PPIF | THRB | |
| CDA | FRS2 | MAP2K1 | PPIP5K2 | TIGD6 | |
| CDC25A | FTSJ2 | MAP2K2 | PPM1D | TIMP3 | |
| CDC25B | FUBP1 | MAP2K3 | PPM1E | TKT | |
| CDC73 | FUS | MAP2K4 | PPP2CA | TLR2 | |
| CDH1 | FYN | MAP2K7 | PPP2CB | TLR4 | |
| CDH19 | FZD1 | MAP3K1 | PPP2R1A | TM6SF1 | |
| CDH8 | G6PC | MAP3K13 | PPP2R1B | TMEM127 | |
| CDK1 | GABBR1 | MAP3K14 | PPP2R5D | TMEM170A | |
| CDK10 | GABBR2 | MAP3K4 | PPP6C | TMEM51 | |
| CDK12 | GABRA6 | MAP3K5 | PRDM1 | TMEM67 | |
| CDK2 | GABRP | MAP3K7 | PRDM2 | TMEM99 | |
| CDK4 | GAK | MAP4K3 | PREP | TMPRSS15 | |
| CDK6 | GALE | MAP4K5 | PREX2 | TMPRSS2 | |
| CDK7 | GALNS | MAPK1 | PRF1 | TMX2-CTNND1 | |
| CDK8 | GALNT12 | MAPK11 | PRKACA | TNFAIP3 | |
| CDKL3 | GALNT14 | MAPK3 | PRKAR1A | TNFRSF10B | |
| CDKN1A | GANC | MAPKAP1 | PRKCB | TNFRSF10D | |
| CDKN1B | GAPDH | MARK2 | PRKCI | TNFRSF11A | |
| CDKN1C | GAPDHS | MAX | PRKDC | TNFRSF11B | |
| CDKN2A | GARS | MBD4 | PROKR2 | TNFRSF14 | |
| CDKN2B | GATA1 | MCL1 | PRPF39 | TNFRSF19 | |
| CDKN2C | GATA2 | MCM4 | PRSS1 | TNFSF13B | |
| CDO1 | GATA3 | MDH2 | PRSS8 | TNFSF14 | |
| CEBPA | GATA6 | MDM2 | PTCH1 | TNKS | |
| CENPF | GCK | MDM4 | PTEN | TNNC1 | |
| CEP120 | GDF7 | MED12 | PTGES | TNRC18 | |
| CEP57 | GDNF | MED12L | PTGR1 | TNRC6A | |
| CFH | GEMIN4 | MED19 | PTGS2 | TNRC6B | |
| CHD1 | GGCT | MED23 | PTK2 | TOMM40L | |
| CHD2 | GGH | MEF2B | PTPN1 | TOP1 | |
| CHD4 | GLB1 | MEF2BNB- | PTPN11 | TOP2A | |
| | | MEF2B | | | |
| CHEK1 | GLI1 | MEIS1 | PTPN22 | TOP2B | |

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES

1. Paiva B, van Dongen J J, Orfao A. New criteria for response assessment: role of minimal residual disease in multiple myeloma. Blood. 2015; 125(20):3059-3068.

2. Brüggemann M, Raff T, Kneba M. Has MRD monitoring superseded other prognostic factors in adult ALL? Blood. 2012; 120(23):4470-4481.

3. Abbosh C, Birkbak N J, Swanton C. Early stage NSCLC—challenges to implementing ctDNA-based screening and MRD detection. Nat Rev Clin Oncol. 2018; 15(9):577-586.

4. Han X, Wang J, Sun Y. Circulating tumor DNA as biomarkers for cancer detection. Genomics Proteomics Bioinformatics. 2017; 15(2):59-72.

5. Abbosh C, Birkbak N J, Wilson G A, et al. Phylogenetic ctDNA analysis depicts early-stage lung cancer evolution. Nature. 2017; 545(7655):446-451.

6. Sethi H, Salari R, Navarro S, et al. Analytical validation of the Signatera™ RUO assay, a highly sensitive patient-specific multiplex PCR NGS-based noninvasive cancer recurrence detection and therapy monitoring assay. In: Proceedings from the American Association for Cancer Research Annual Meeting; Apr. 17, 2018; Chicago, Ill. Abstract 4542.

7. Reinert T, Henriksen T V, Rasmussen M H, et al. Serial circulating tumor DNA analysis for detection of residual disease, assessment of adjuvant therapy efficacy and for early recurrence detection in colorectal cancer. Poster presented at: ESMO 2018 Congress; Oct. 19-23, 2018; Munich, Germany. Abstract 5433.

8. Birkenkamp-Demtröder K, Christensen E, Sethi H, et al. Sequencing of plasma cfDNA from patients with locally advanced bladder cancer for surveillance and therapeutic efficacy monitoring. Poster presented at: ESMO 2018 Congress; Oct. 19-23, 2018; Munich, Germany. Abstract 5964

9. Coombes R C, Armstrong A, Ahmed S, et al. Early detection of residual breast cancer through a robust, scalable and personalized analysis of circulating tumour DNA (ctDNA) antedates overt metastatic recurrence. Poster presented at: San Antonio Breast Cancer Symposium; December 4-8, 2018; San Antonio, Tex. Abstract 1266.

10. Reiman A, Kikuchi H, Scocchia D, et al. Validation of an NGS mutation detection panel for melanoma. BMC Cancer. 2017; 17:150.

11. Simen B B, Yin L, Goswami C P, et al. Validation of a next-generation-sequencing cancer panel for use in the clinical laboratory. Arch Pathol Lab Med. 2015; 139(4):508-517

12. Singh R R, Patel K P, Routbort M J, et al. Clinical massively parallel next-generation sequencing analysis of 409 cancer-related genes for mutations and copy number variations in solid tumours. Br J Cancer. 2014; 111(10):2014-2023.

13. Domínguez-Vigil I G, Moreno-Martinez A K, Wang J Y, Roehrl M H A, Barrera-Saldaña H A. The dawn of the liquid biopsy in the fight against cancer. Oncotarget. 2018; 9:2912-2922. doi: 10.18632/oncotarget .23131.

14. Lanman R B, Mortimer S A, Zill O A, et al. Analytical and clinical validation of a digital sequencing panel for quantitative, highly accurate evaluation of cell-free circulating tumor DNA. PLoS One. 2015;10(10) :e 0140712. doi: 10.1371/journal.pone.0140712.

15. Plagnol V, Woodhouse S, Howarth K, et al. Analytical validation of a next generation sequencing liquid biopsy assay for high sensitivity broad molecular profiling. PLoS One. 2018;13(3):e 0193802. doi: 10.1371/journal. pone.0193802.

16. Foundation Medicine, Inc. Foundation Medicine Web site.

https://www.foundationmedicine.com/genomic-testing/foundation-one-liquid. Accessed Mar. 18, 2019.

17. Oncomine™ lung cfDNA assay. Thermo Fisher Scientific Web site. https:// www.thermofisher.com/order/catalog/product/A31149. Accessed Mar. 18, 2019.

18. Zimmermann B, Salari R, Swenerton R. Personalized Liquid Biopsy: Patient-Specific Non-Invasive Cancer Recurrence Detection and Therapy Monitoring. Paper presented at: 10th Circulating Nucleic Acids in Plasma and Serum (CNAPS) International Symposium; Sep. 20-22 , 2017; Montpellier, France.

19. Costello M, Pugh T J, Fennell T J, et al. Discovery and characterization of artifactual mutations in deep coverage targeted capture sequencing data due to oxidative DNA damage during sample preparation. Nucleic Acids Res. 2013; 41:e 67.

20. Chen G, Mosier S, Gocke C D, Lin M T, Eshleman J R. Cytosine deamination is a major cause of baseline noise in next-generation sequencing. Mol Diagn Ther. 2014; 18:587-593.

21. Newman A M, Lovejoy A F, Klass D J, et al. integrated digital error suppression for improved detection of circulating ttumor DNA. Nat Biotechnol. 2016; 34:547-555.

22. Early Detection of Molecular Residual Disease in Localized Lung Cancer by Circulating Tumor DNA Profiling. Cancer Discov. 2017 December; 7(12): 1394-1403. doi:10.1158/2159-8290.CD-17-0716.

23. An ultrasensitive method for quantitating circulating tumor DNA with broad patient coverage. Nat Med. 2014 May; 20(5): 548-554. doi:10.1038/nm.3519.

24. Zviran A, Schulman R C, Shah M, et al. Genome-wide cell-free DNA mutational integration enables ultra-sensitive cancer monitoring[J]. Nature medicine, 2020, 26(7):1-11.

1. A method for determining the minimal residual cancer status of an individual comprising:
a) selecting a panel of loci comprising human genomic regions that may host mutated genes in a particular type of solid tumor;
b) referencing a database of baseline measures of sequence information for the panel of loci;
c) preparing at least one mathematical distribution of sequence information at one or more locus based on the database of step (b), wherein a first portion of the baseline measures at a locus is classified as not exhibiting variation and a second portion of the baseline measures at the locus is classified as exhibiting variation, wherein the second portion of the baseline measures is statistically fitted and combined with the first portion of baseline measures;
d) obtaining tumor sample DNA sequence information collected from a tumor sample from the individual and identifying one or more genomic variants within the selected panel of loci;
e) obtaining extracellular DNA sequence information for the panel of loci from the individual, wherein the sequence information is collected from a plasma sample from the individual, wherein the plasma sample comprises extracellular DNA;
f) comparing the sequence information of step (e) to at least one corresponding distribution of step (c) for one or more genomic variants of step (d), wherein the comparison determines probabilities that differences exist at the one or more genomic variants between the extracellular DNA sequence information of the individual and the corresponding baseline measures of step (b), thereby providing at least one probability of genomic variant level significance;
g) combining the genomic variant level significance probabilities into a combined sample level probability score and
h) determining that the individual has a positive status for minimal residual cancer if the p-value of the combined sample level probability score of step (g) is equal to or less than a threshold value.

2. (canceled)

3. A method for determining the minimal residual cancer status of an individual comprising:
a) selecting a panel of loci comprising human genomic regions that may host mutated genes in a particular type of solid tumor;
b) referencing a database of baseline measures of sequence information for the panel of loci;
c) preparing at least one mathematical distribution of sequence information at one or more locus based on the database of step (b), wherein a first portion of the baseline measures at a locus is classified as not exhibiting variation and a second portion of the baseline measures at the locus is classified as exhibiting variation, wherein the second portion of the baseline measures is statistically fitted and combined with the first portion of baseline measures;
d) obtaining tumor sample DNA sequence information collected from a tumor sample from the individual and identifying one or more genomic variants within the selected panel of loci;
e) obtaining extracellular DNA sequence information for the panel of loci from the individual, wherein the sequence information is collected from a plasma sample from the individual, wherein the plasma sample comprises extracellular DNA;
f) comparing the sequence information of step (e) to at least one corresponding distribution of step (c) for at least one genomic variants of step (d), wherein the comparison determines a probability that a difference exists at the one or more genomic variants between the extracellular DNA sequence information of the individual and the corresponding baseline measures of step (b), thereby providing at least one probability of genomic variant level significance; and
g) determining that the individual has a positive status for minimal residual cancer if the p-value of at least one genomic variant of step (f) is equal to or less than a threshold value.

4. (canceled)

5. A method for determining the minimal residual cancer status of an individual comprising:
a) selecting a panel of loci comprising human genomic regions that may host mutated genes in a particular type of solid tumor;
b) referencing a database of baseline measures of sequence information for the panel of loci;
c) preparing at least one mathematical distribution of sequence information at one or more locus based on the database of step (b), wherein any variation exhibited by the baseline measures is conformed to a binomial distribution;
d) obtaining tumor sample DNA sequence information collected from a tumor sample from the individual and identifying one or more genomic variants within the selected panel of loci;
e) obtaining extracellular DNA sequence information for the panel of loci from the individual, wherein the sequence information is collected from a plasma sample from the individual, wherein the plasma sample comprises extracellular DNA;
f) comparing the sequence information of step (e) to at least one corresponding distribution of step (c) for one or more genomic variants of step (d), wherein the comparison determines probabilities that differences exist at the one or more genomic variants between the extracellular DNA sequence information of the individual and the corresponding baseline measures of step (b), thereby providing at least one probability of genomic variant level significance;
g) combining the genomic variant level significance probabilities into a combined sample level probability score; and
h) determining that the individual has a positive status for minimal residual cancer if the p-value of the combined sample level probability score of step (g) is equal to or less than a threshold value.

6. (canceled)

7. A method for determining the minimal residual cancer status of an individual comprising:
a) selecting a panel of loci comprising human genomic regions that may host mutated genes in a particular type of solid tumor;
b) referencing a database of baseline measures of sequence information for the panel of loci;
c) preparing at least one mathematical distribution of sequence information at one or more locus based on the database of step (b), wherein any variation exhibited by the baseline measures is conformed to a binomial distribution;
d) obtaining tumor sample DNA sequence information collected from a tumor sample from the individual and identifying one or more genomic variants within the selected panel of loci;
e) obtaining extracellular DNA sequence information for the panel of loci from the individual, wherein the sequence information is collected from a plasma sample from the individual, wherein the plasma sample comprises extracellular DNA;
f) comparing the sequence information of step (e) to at least one corresponding distribution of step (c) for at least one genomic variants of step (d), wherein the comparison determines a probability that a difference exists at the one or more genomic variants between the extracellular DNA sequence information of the individual and the corresponding baseline measures of step (b), thereby providing at least one probability of genomic variant level significance; and
g) determining that the individual has a positive status for minimal residual cancer if the p-value of at least one genomic variant of step (f) is equal to or less than a threshold value.

8. (canceled)

9. The method of claim 1, wherein the fitting is performed by application of a statistical model selected from the group consisting of a beta-distribution, a gamma-distribution, a Weibull-distribution and any combination thereof.

10. The method of claim 1, wherein combining the genomic variant level significance probabilities into a combined sample level probability score comprising application of the formula Pₛₐₘₚₗₑ=Cₘ^{k}ΠPᵢ, wherein m of the combination coefficient (C) represents the number of variants tracked and k represents the number of variants that have passed a variant level threshold, wherein only the variant level significance probabilities that have passed the variant level threshold are included in the Pi multiplication.

11. The method of claim 1, wherein sequence information for the individual and sequence information comprised by the baseline measures was collected by PCR or hybridization.

12. The method of claim 11, wherein the sequence information was collected by PCR.

13. The method of claim 11, wherein the sequence information was collected by hybridization.

14. The method of claim 1, wherein the extracellular DNA sequence information for the panel comprises features selected the group consisting of mapping quality, base quality, position depth, variant supported molecules, fragment size, reads pair concordance, distance from the fragment end, and single/duplex consensus.

15. The method of claim 1, wherein the sequence information collected from the plasma sample comprises features selected the group consisting of mapping quality, base quality, position depth, variant supported molecules, fragment size, reads pair concordance, distance from the fragment end, and single/duplex consensus.

16. The method of claim 14, wherein the comparison of step (f) comprises authentication of at least one feature.

17. The method of claim 1, wherein step (b) comprises sequence information obtained for a corresponding panel of loci for extracellular DNA from plasma samples from individuals classified as negative for the cancer.

18. The method of claim 1, wherein step (b) comprises sequence information obtained by sequencing tumor and plasma samples from individuals having cancer with the same type of solid tumor, wherein mathematical information for genomic variants within the selected panel of loci identified in the tumor is subtracted from mathematical information for genomic variants within the selected panel of loci in corresponding plasma sample to simulate individuals negative for the cancer.

19. The method of claim 1, wherein the comparison of step (f) comprises application of a Monte Carlo simulation.

20. The method of claim 1, wherein the comparison of step (f) comprises application of a statistical test based on an expectation set by a mathematical distribution in step (c).

21. The method of claim 1, wherein in step (c), three mathematical distributions of sequence information are prepared, one for each substitution at each base position of the locus.

22. The method of claim 1, wherein in step (c) at least one locus exhibits an insertion or deletion and further wherein, one mathematical distribution of sequence information is prepared, one for each insertion or deletion at the locus.

23. The method of claim 1, wherein noise is reduced by limiting tracking to tracking of tumor tissue-specific mutations only in plasma.

24. The method of claim 10, wherein m≥1.

25. The method of claim 1, wherein the panel of loci comprises at least one mutation known to be associated with the type of cancer for which minimal residual cancer status is determined.

26. The method of claim 1, wherein the cancer is selected from the group consisting of lung cancer, breast cancer, prostate cancer, colon cancer, melanoma, bladder cancer, non-Hodgkin's lymphoma, renal cancer, endometrial cancer, leukemia, pancreatic cancer, thyroid cancer, and liver cancer.

27. The method of claim 1, wherein the individual has previously received treatment for cancer.

28. The method of claim 27, wherein the treatment for cancer was selected from the group consisting of a drug, a radiation treatment, a surgery and any combination thereof.

29. A computer-implemented method for determining the minimal residual cancer status of an individual according to the method of claim 1, wherein one or more of steps (b), (c), (f), (g) and (h) are computed with a computer system.

30. A computer-implemented method for determining the minimal residual cancer status of an individual according to the method of claim 3, wherein one or more of steps (b), (c), (f), and (g) are computed with a computer system.

31. A program storage device readable by a computer, tangibly embodying a program of instructions executable by the computer to perform the method steps of claim 1.

32. A computing system for determining the minimal residual cancer status of an individual comprising: a memory for storing programmed instructions; a processor configured to execute the programmed instructions to perform the methods steps of claim 1.

33. A non-transitory, computer readable media with instructions stored thereon that are executable by a processor to perform the methods steps of claim 1.
